(19) United States
(12) Patent Application Publication
(10) Pub. No.: US 2025/0032627 A1
(43) Pub. Date: Jan. 30, 2025

(54) THERAPEUTIC SEALANTS BASED UPON DRUG CONJUGATES

(71) Applicants: The Johns Hopkins University, Baltimore, MD (US); Integra LifeSciences Corporation, Princeton, NJ (US)

(72) Inventors: Honggang CUI, Lutherville, MD (US); Han Wang, Baltimore, MD (US); Tian Xu, Baltimore, MD (US); Steve Bennett, Princeton, NJ (US)

(21) Appl. No.: 18/767,316

(22) Filed: Jul. 9, 2024

Related U.S. Application Data
(60) Provisional application No. 63/513,593, filed on Jul. 14, 2023.

(51) Int. Cl. A61K 47/60; A61K 31/4745; A61K 47/02; A61P 35/00

(52) U.S. Cl. CPC A61K 47/60; A61K 31/4745; A61K 47/02; A61P 35/00

(57) ABSTRACT

A series of peptide-drug conjugates that react with multi-arm polyethylene glycol (PEG) to form chemically cross-linked hydrogels and their use as both a sealant and a therapeutic depot is disclosed.

### CROSS-REFERENCE TO RELATED APPLICATIONS

This application claims priority to U.S. Application No. 63/513,593 filed on Jul. 14, 2023, the contents of which are herein incorporated by reference.

### BACKGROUND

Hydrogels can be used in a variety of medical applications in vivo, such as sealing, adhesion prevention, and drug delivery. One hydrogel-based composition known in the art is a 4-arm, 20-kDa polyethylene glycol crosslinked with trilysine, which can be used, for example, to prevent leakage of cerebrospinal fluid from dural sutures during spinal surgery. Such hydrogel-based compositions can be hydrolyzed and absorbed over, for example, a 4- to 8-week period. Other similar formulations using a lower molecular weight polyethylene glycol have been reported to exhibit less swelling than the original formulation. These formulations also are degraded by hydrolysis and reabsorbed over a 9- to 12-week period. In both cases, the hydrogel is thought to adhere to tissue by mechanical means. Despite the availability of these hydrogel-based compositions, there is a need for therapeutic sealants capable of delivering a therapeutic agent to a targeted area of a subject in need of treatment.

### SUMMARY

In some aspects, the presently disclosed subject matter provides a composition of formula (I):

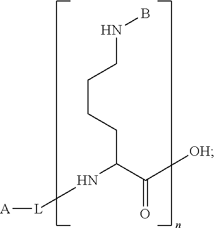

wherein: n is an integer selected from 2, 3, 4, and 5; A is a therapeutic agent; L is a linker; and each B is independently a multi-arm polyethylene glycol (PEG) moiety, wherein each B can be the same or different.

In certain aspects, the composition of formula (I) is selected from:

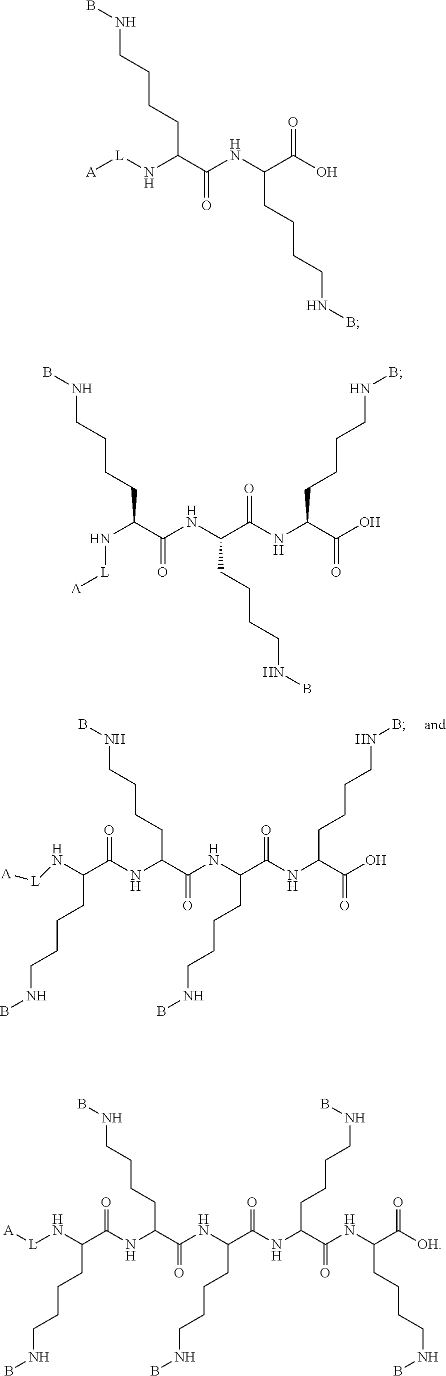

In particular aspects, the composition of formula (I) comprises:

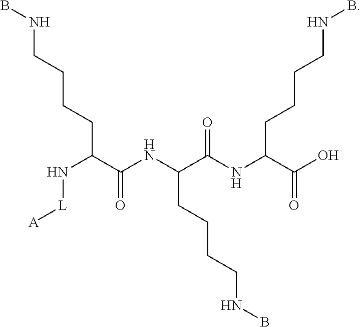

In certain embodiments, the composition of formula (I) comprises:

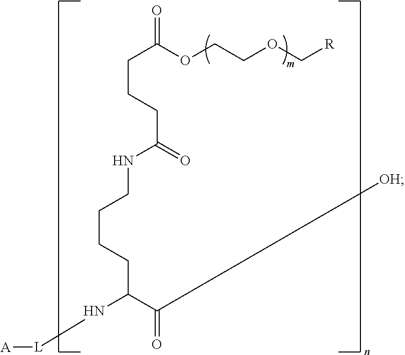

wherein: each m is independently an integer selected from 1 to 1,000; each n is independently an integer selected from 1 to 2,000; and each R independently comprises a core of a multi-arm PEG moiety.

In more certain aspects, the composition of formula (I) comprises:

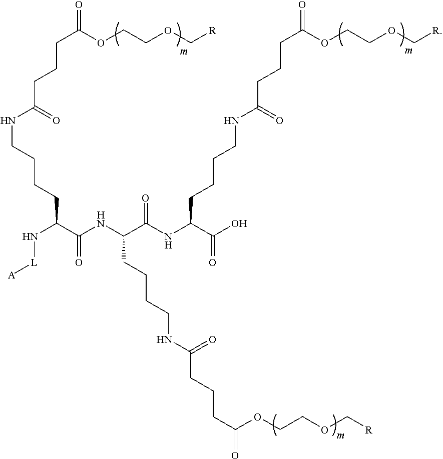

In certain aspects, the multi-arm PEG moiety has between 2 and 10 arms. In particular aspects, the multi-arm PEG moiety is selected from a 2-arm PEG moiety, a 4-arm PEG moiety, and an 8-arm PEG moiety. In particular aspects, the multi-arm PEG moiety further comprises an end group selected from —OH, —SH, —NH₂, —N₃, alkoxyl, halogen, —CH═CH₂, —C(CH₃)═CH₂, —C≡CH, —COOH, a succinimidyl ester, and a maleimide.

In certain aspects, the linker comprises a biodegradable linker. In particular aspects, the biodegradable linker is selected from:

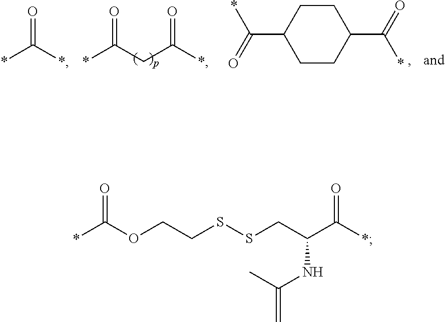

wherein * denotes a point of attachment of the linker to the therapeutic agent and the lysine moiety and p is an integer selected from 1, 2, 3, 4, 5, 6, 7, 8, 9, 10, 11, and 12. In particular aspects, the linker comprises a succinate moiety having a chemical structure of:

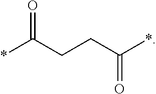

In certain aspects, the composition of formula (I) comprises:

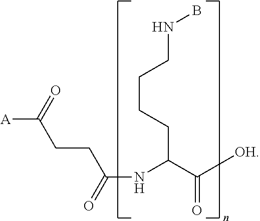

In certain aspects, the therapeutic agent comprises a hydrophobic or a hydrophilic drug. In more certain aspects, the drug is selected from an anti-cancer drug, an antibiotic, an antiviral agent, a hemostatic agent, and an anesthetic. In particular aspects, the antibiotic is selected from a penicillin, a macrolide, a cephalosporin, a fluoroquinolone, a beta-lactam, a tetracycline, trimethoprim-sulfamethoxazole, a urinary anti-infective, a lincosamide, and combinations thereof. In more particular aspects, the antibiotic is selected from penicillin, amoxicillin, azithromycin, erythromycin, cephalexin, cefdinir, ciprofloxacin, levofloxacin, tetracycline, doxycycline, nitrofurantoin, and clindamycin. In particular aspects, the anesthetic is selected from bupivacaine, lidocaine, proparacaine, tetracaine, dibucaine, benoxinate, ropivacaine, articaine, carbocaine, marcaine, mepivacaine, polocaine, prilocaine, sensorcaine, and septocaine. In particular aspects, the anti-cancer drug is selected from camptothecin (CPT), paclitaxel, docetaxel, tamoxifen, and analogues and combinations thereof. In more particular aspects, the anti-cancer drug comprises camptothecin or an analogue thereof. In certain aspects, the analogue of camptothecin is selected from topotecan, irinotecan (CPT-11), silatecan (DB-67, AR-67), cositecan (BNP-1350), exatecan, lurtotecan, gimatecan (ST1481), belotecan (CKD-602), and rubitecan.

In certain aspects, the composition has a chemical structure selected from:

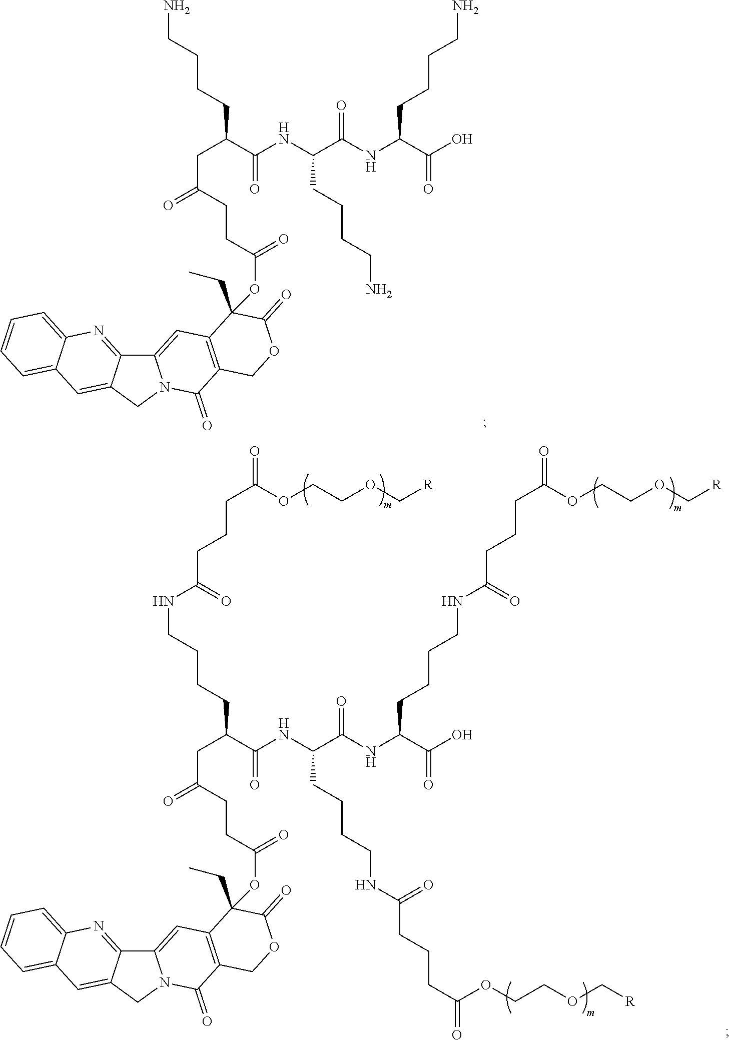

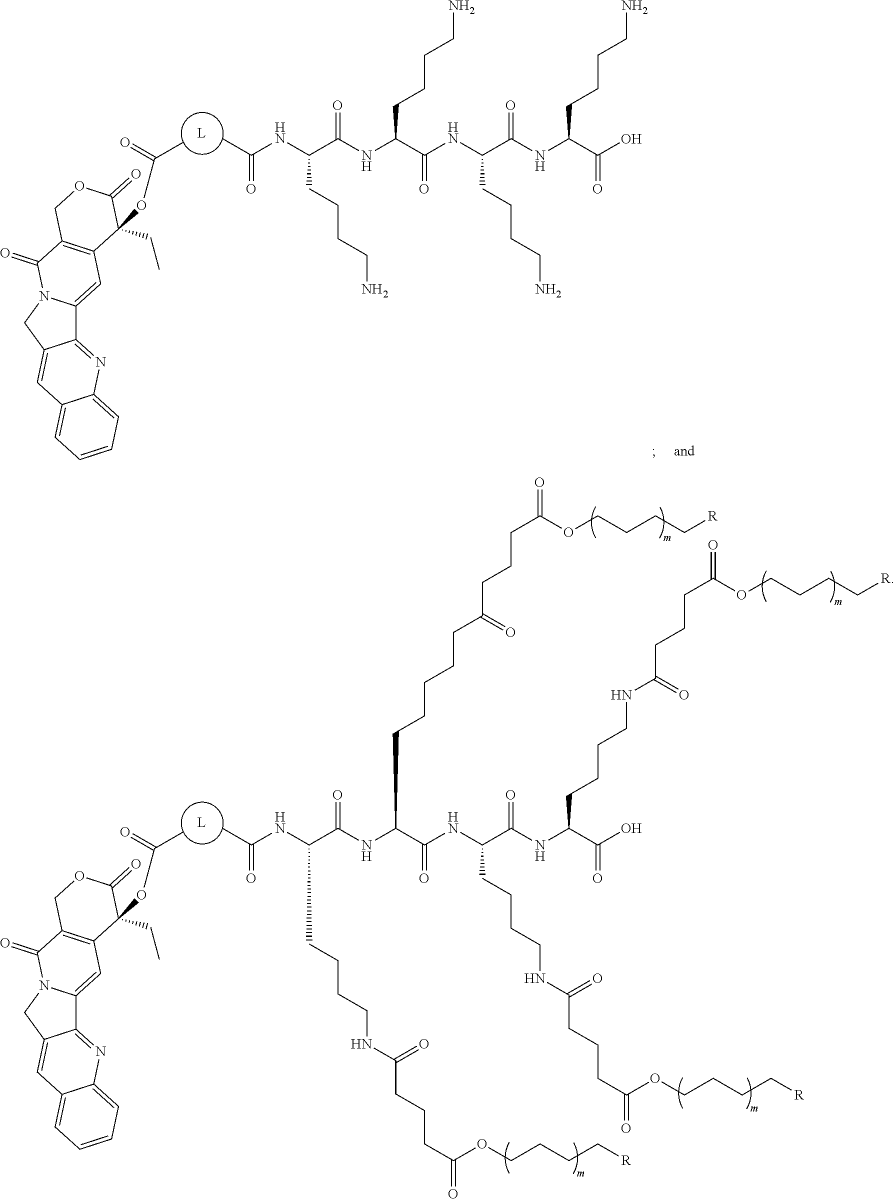

In some aspects, the composition has a chemical structure selected from:

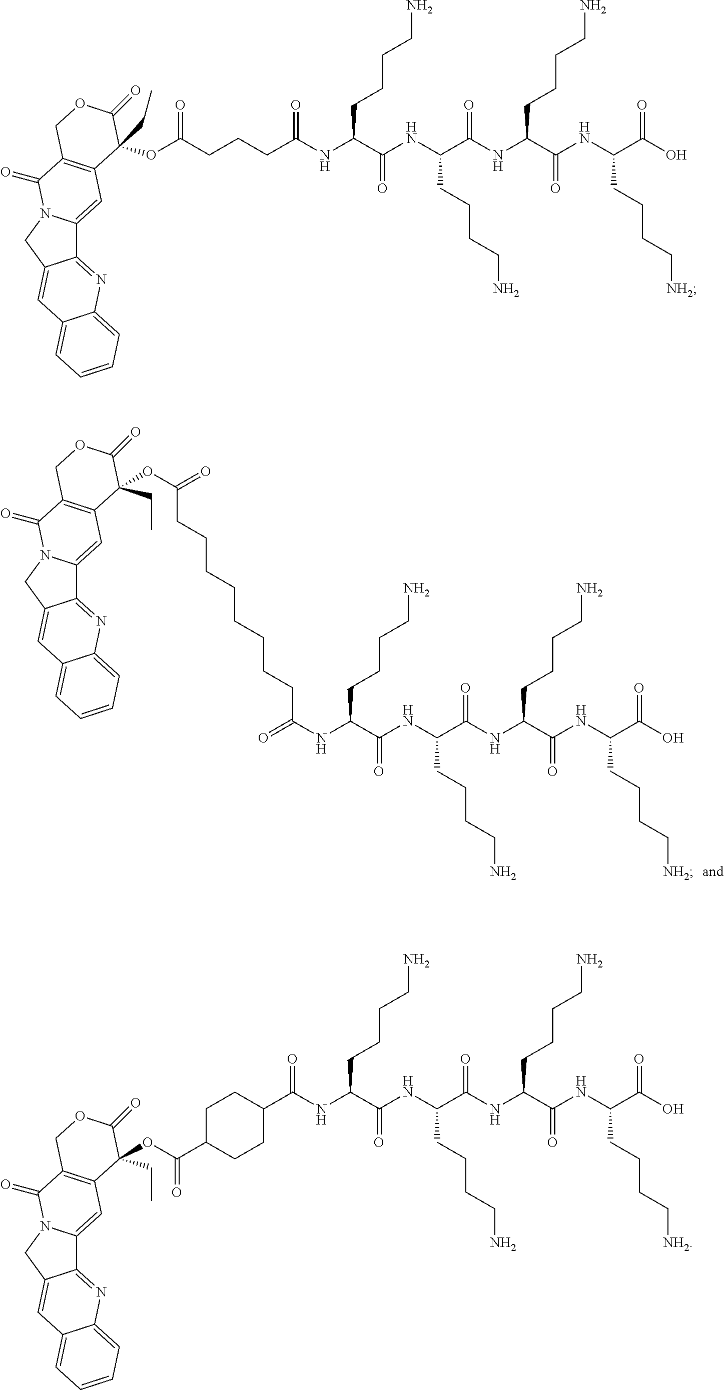 [SMILES: CC[C@]1(OC(=O)CCCC(=O)N[C@@H](CCCCN)C(=O)N[C@@H](CCCCN)C(=O)N[C@@H](CCCCN)C(=O)N[C@@H](CCCCN)C(=O)O)C(=O)OCc2c1cc1n(c2=O)Cc2cc3ccccc3nc2-1 ; CC[C@]1(OC(=O)CCCCCCCCC(=O)N[C@@H](CCCCN)C(=O)N[C@@H](CCCCN)C(=O)N[C@@H](CCCCN)C(=O)N[C@@H](CCCCN)C(=O)O)C(=O)OCc2c1cc1n(c2=O)Cc2cc3ccccc3nc2-1 ; CC[C@@]1(OC(=O)C2CCC(C(=O)N[C@@H](CCCCN)C(=O)N[C@@H](CCCCN)C(=O)N[C@@H](CCCCN)C(=O)N[C@@H](CCCCN)C(=O)O)CC2)C(=O)OCc2c1cc1n(c2=O)Cc2cc3ccccc3nc2-1]

In certain aspects, the presently disclosed composition further comprises a buffer. In particular aspects, the buffer comprises a borate buffer. In more particular aspects, the borate buffer comprises a nano-confined borate buffer.

In other aspects, the presently disclosed subject matter provides a method for treating a disease, disorder, or condition in a subject, the method comprising administering a composition described herein to a subject in need of treatment thereof. In certain aspects, the disease, disorder, or condition is selected from a cancer, an infection, and an inflammation. In particular aspects, the cancer is selected from breast cancer, ovarian cancer, colon cancer, stomach cancer, non-small cell lung cancer (NSCLC), a glioblastoma, and Kaposi sarcoma.

In certain aspects, the therapeutic agent is released by ester hydrolysis in vivo.

In other aspects, the presently disclosed subject matter provides a composition described herein and instructions for use. In particular aspects, the kit further comprises one or more components selected from one or more solvent or buffers, one or more vials, one or more syringes, and instructions for use.

In other aspects, the presently disclosed subject matter provides a sealant comprising the composition described herein.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying drawings, which are not necessarily drawn to scale, and wherein:

FIG. 1A, FIG. 1B, FIG. 1C, and FIG. 1D show (FIG. 1A, FIG. 1B) components of the presently disclosed sealant system; (FIG. 1C) a representative method for preparing the presently disclosed sealant system incorporating, for example, a representative small molecular hydrophobic anticancer drug camptothecin (CPT) as a model drug; and (FIG. 1D) shows a cross-linked system formed through crosslinking CPT-K3 with a multi-arm PEG-NHS ester;

FIG. 2A, FIG. 2B, and FIG. 2C show a representative synthesis scheme for the preparation of CPT-K3 (FIG. 2A); an HPLC trace of the synthesized CPT-K3 product (FIG. 2B); and an ESI-MS spectrum of the synthesized CPT-K3 product (FIG. 2C);

FIG. 3A and FIG. 3B demonstrate that CPT-K3 remains soluble and stable in a borate buffer solution. FIG. 3A shows that CPT-K3 exhibits good solubility in borate buffer and is stable in 75-mM borate buffer for at least 24 hours. FIG. 3B indicates that CPT-K3 did not assemble into a nanostructure in either PBS or water;

FIG. 4A, FIG. 4B, and FIG. 4C demonstrate that CPT-K3 forms a cross-linked hydrogel. FIG. 4A shows photographs demonstrating that CPT-K3 forms a hydrogel after crosslinking in a 1:1 stoichiometry; FIG. 4B shows an average swelling rate of 120% in 1.0 to 1.2 CPT-K3 equivalent; and FIG. 4C shows CPT release monitored by HPLC;

FIG. 5 demonstrates the release of CPT by ester hydrolysis;

FIG. 6A, FIG. 6B, and FIG. 6C demonstrate that color shift in 5-FAM hydrogel indicated gradual decreased local pH over hydrogel degradation;

FIG. 7A, FIG. 7B, and FIG. 7C demonstrate CPTK3-8A release with different borate concentrations. FIG. 7A is a table providing the borate equivalent, excess borate equivalent, excess borate (mM), and CPTK3 (mM). FIG. 7B is a plot of the swelling (%) vs. excess borate equivalent. FIG. 7C is a plot of the cumulative release (%) per day of excess borate equivalent;

FIG. 8A and FIG. 8B demonstrate that drug release in CPTK4/A4 hydrogel is controlled by nano-confined borate. FIG. 8A is a plot of the cumulative release (%) vs. day. FIG. 8B is a plot of the burst release (%) vs. half-life (day). Each graph depicts borate at 0.3, 0.4, 0.5, 0.6, 0.7, 0.8, 0.9, and 1.0. Sodium borate at a concentration of 75 mM serves as a reference point for one equivalent. To investigate the impact of varying borate concentrations on the hydrogel's properties, a series of hydrogels with different borate levels were prepared. Each of these levels is represented as a certain borate equivalent, where the term “borate equivalent” refers to the molar ratio of borate to the reference 75-mM sodium concentration. This approach enables a systematic comparison of hydrogels with distinct borate compositions, facilitating the optimization of the formulation;

FIG. 9A and FIG. 9B demonstrate that nano-confined borate controls hydrogel degradation: (FIG. 9A) CPT-K4/A4. (FIG. 9B) CPT-K4/A8;

FIG. 10A and FIG. 10B demonstrate that CPT-K3 exhibited in vitro cytotoxicity against U87 glioblastoma cells. FIG. 10A is a table providing the IC50 of CPT and CPT-K3. FIG. 10B is a plot of cell viability (%) vs. concentration;

FIG. 11A, FIG. 11B, and FIG. 11C demonstrate the in vitro cytotoxicity of the presently disclosed sealant system against U87 cells. FIG. 11A is a schematic showing a representative experimental setup for measuring the cytotoxicity of the presently disclosed sealant system. FIG. 11B is a plot of the cell viability (%) vs. dose of the presently disclosed sealant system, including 25 mg, 50 mg, 100 mg, and PBS. FIG. 11C is a plot of the cell viability vs. incubation time for two hydrogel-based compositions known in the art and CPT;

FIG. 12 shows the in vivo therapeutic effect of CPT-K4/A4 against U87 tumor;

FIG. 13A and FIG. 13B demonstrate the in vivo therapeutic effect against U87 tumor. FIG. 13A is a plot of the tumor volume (mm³) vs. time (day). FIG. 13B is a plot of body weight (g) vs. time (day);

FIG. 14 shows the safety and biocompatibility of the presently disclosed sealant system. Shown are plots of the ALP (U/L), AST (U/L), ALT (U/L), total bilirubin (mg/dL), BUN (mg/dL), and creatinine (mg/dL) at day 15 and day 30 compared to PBS;

FIG. 15 shows the serum chemistry of CPT-K4/4A. Shown are plots of the ALP (U/L), AST (U/L), ALT (U/L), total bilirubin (mg/dL), BUN (mg/dL), and creatinine (mg/dL) at day 15 and day 30 compared to PBS;

FIG. 16 shows the serum chemistry of CPT-K4/8A. Shown are plots of the ALP (U/L), AST (U/L), ALT (U/L), total bilirubin (mg/dL), BUN (mg/dL), and creatinine (mg/dL) at day 15 and day 30 compared to PBS;

FIG. 17 shows the total blood cell count. Shown are plots of RBC (M/μL), neutrophil (K/μL), lymphocytes (K/μL), monocytes (K/μL), and WBC (K/μL) for CPTK4/A4, CPTK4/A8, and PBS. CPTK4/A4 @ day 14. CPTK4/A8 @ day 60;

FIG. 18 shows H&E-stained tissue sections of heart, kidney, liver, lung, spleen, and skin for CPT-K4/A4 at day 15 and day 30 and CPT-K4/A8 at day 30 and day 60 compared to PBS;

FIG. 19 shows illustrative examples for optimizing drug release with linker design;

FIG. 20 shows representative camptothecin (CPT) prodrug synthesis;

FIG. 21A demonstrates release of CPT in vitro;

FIG. 21B a representative generic structure of the presently disclosed sealant system with L in this embodiment representing a hydrophobic linker;

FIG. 21C shows representative compounds having a hydrophobic linker, e.g., CPT-c5-K4 with a five carbon (c5) linker, CPT-c10-K4 with a ten carbon (c10) linker; and CPT-mC-K4 with a cyclohexylene (mC) linker;

FIG. 22A, FIG. 22B, and FIG. 22C show MALDI-Tof MS of (FIG. 22A) CPT-c5-K4; (FIG. 22B) CPT-c10-K4; and (FIG. 22C) CPT-mC-K4; and

FIG. 23A and FIG. 23B show hydrogel formation of CPT-L-K4 with 20k 8A PEG NHS, for various linkers, L, including linkers referred to herein as c5, c10, and mC. FIG. 23A shows photographs demonstrating that CPT-mC-K4/8A formed brittle hydrogels, possibly due to the rigid mC linker. FIG. 23B is a graph demonstrating that low swelling ratio (<10) were observed in all hydrogels.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

The presently disclosed subject matter provides a series of peptide-drug conjugates that react with multi-arm polyethylene glycol (PEG) to form chemically cross-linked hydrogels. The hydrogel systems are able to function as both a sealant and a therapeutic depot.

The presently disclosed system allows for facile-tuning of release and swelling rate because the pharmacokinetics of the active drug is controlled by the hydrolysis rate of ester bonds, the number of PEG arms, and the number of lysine arms. Modification of either of these design features can control the dissociation of the drug molecules. The prodrug cleavage rate also can be controlled by the internal pH of the hydrogel depot. This control of the prodrug cleavage rate provides an approach to modulate the pharmacokinetic profiles without changing the molecular design.

In some embodiments, the peptide drug conjugates include an oligolysine peptide backbone and one or more therapeutic molecules conjugated to the N-terminus of the peptide backbone. The oligolysine backbone has the general structure of:

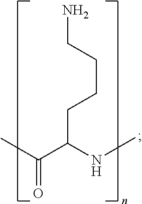

wherein n is an integer selected from 2, 3, 4, and 5. Without wishing to be bound to any one particular theory, it is thought that an increase in the number of lysine sequences can lead to an increase in cross-linking density.

Representative oligolysine sequences suitable for use with the presently disclosed subject matter include, but are not limited to, dilysine, trilysine, tetralysine, and pentalysine, the chemical structures of which are provided immediately herein below:

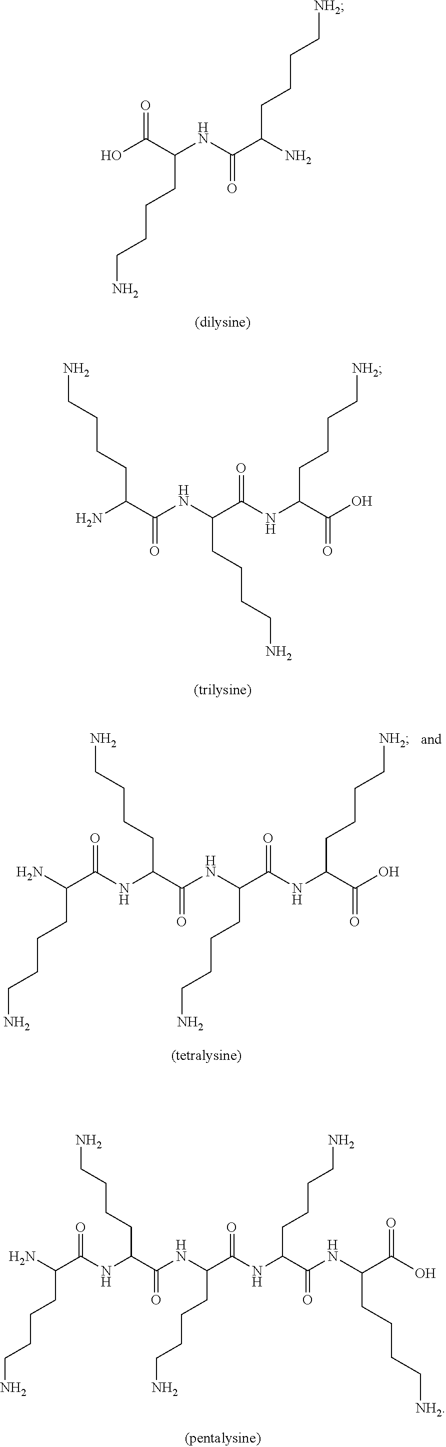 [SMILES: NCCCCC(N)C(=O)NC(CCCCN)C(=O)O ; NCCCCC(N)C(=O)NC(CCCCN)C(=O)NC(CCCCN)C(=O)O ; NCCCCC(N)C(=O)NC(CCCCN)C(=O)NC(CCCCN)C(=O)NC(CCCCN)C(=O)O ; NCCCCC(N)C(=O)NC(CCCCN)C(=O)NC(CCCCN)C(=O)NC(CCCCN)C(=O)NC(CCCCN)C(=O)O]

Both hydrophilic and hydrophobic drugs can be conjugated to the peptide via biodegradable and bio-responsive linkers to achieve good water solubility and improved chemical stability.

More particularly, in some embodiments, the presently disclosed subject matter provides a composition of formula (I):

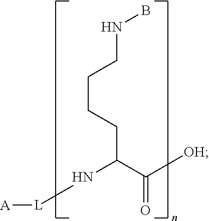

wherein: n is an integer selected from 2, 3, 4, and 5; A is a therapeutic agent; L is a linker; and each B is independently a multi-arm polyethylene glycol (PEG) moiety, wherein each B can be the same or different.

In certain embodiments, the composition of formula (I) is selected from:

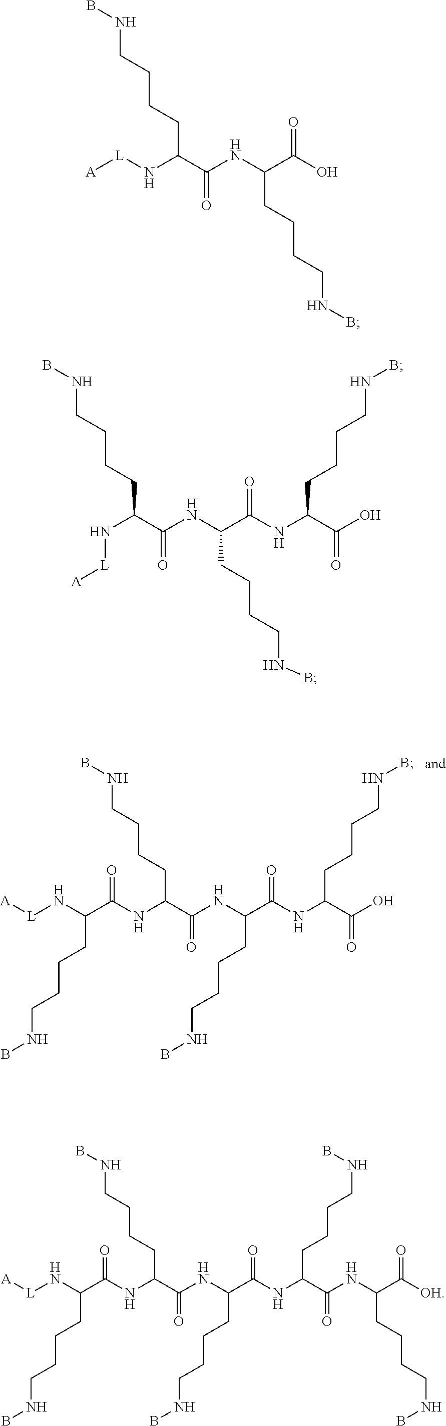

In particular embodiments, the composition of formula (I) comprises:

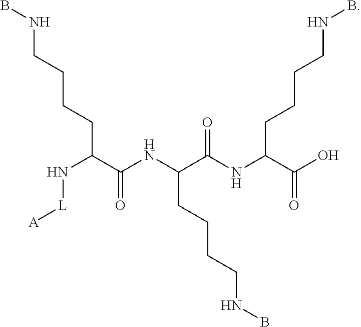

In certain embodiments, the composition of formula (I) comprises:

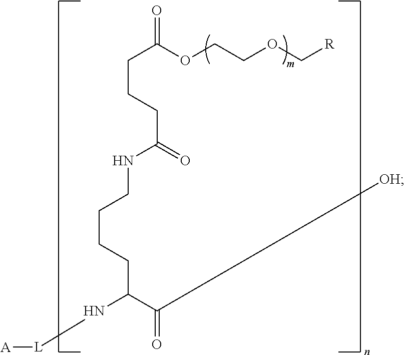

wherein: each m is independently an integer selected from 1 to 1,000; each n is independently an integer selected from 1 to 2,000; and each R independently comprises a core of a multi-arm PEG moiety.

In more certain embodiments, the composition of formula (I) comprises:

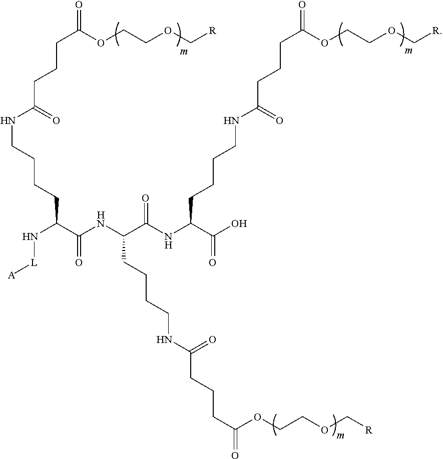

In certain embodiments, the multi-arm PEG moiety has between 2 and 10 arms, including 2, 3, 4, 5, 6, 7, 8, 9, and 10 arms. In particular embodiments, the multi-arm PEG moiety is selected from a 2-arm PEG moiety, a 4-arm PEG moiety, and an 8-arm PEG moiety.

In certain embodiments, the multi-arm PEG units have a number average molecular weight from about 2 kDa and 60 kDa. In particular embodiments, the multi-arm PEG units have a number average molecular weight of about 2 kDa, 5 kDa, 10 kDa, 20 kDa, 40 kDa, and 60 kDa.

In certain embodiments, the multi-arm PEG moiety can be represented as follows:

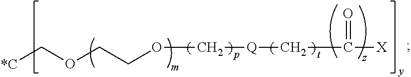

wherein: *C is a central carbon of the core of the multi-arm PEG moiety, Q can be present or absent and when present is O or NH, m is an integer from 1 to 1,000, p and t are each independently an integer selected from 0, 1, 2, 3, 4, 5, 6, 7, and 8, z is 0 or 1, y is an integer selected from 2, 4, and 8, X is selected from —OH, —SH, —NH₂, —N₃, —CH═CH₂, —C(CH₃)═CH₂, —C≡CH, —COOH, halogen, alkoxyl, an isocyanate, a succinimidyl ester, and a malemide.

In particular embodiments, the 4-arm PEG moiety comprises a pentaerythritol core structure and can be represented as follows:

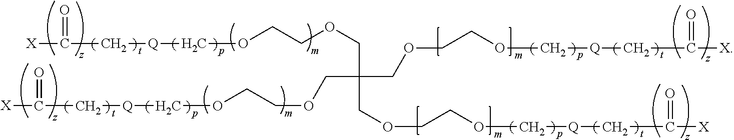

In certain embodiments, the 8-arm PEG moiety can be represented as follows:

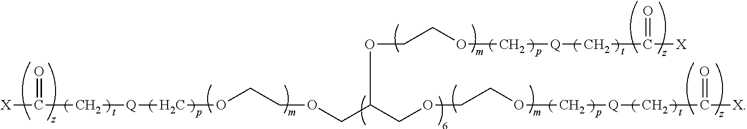

In certain embodiments, for example, the 8-arm polyethylene glycol (PEG) N-hydroxysuccinimide (NHS) ester enables a faster cross-linking reaction with the amine groups in the conjugates. Other suitable polymers include, but are not limited to, polyethylene oxide, polypropylene oxide, polyvinyl alcohol, poly (vinylpyrrolidinone), polylactic acid, polylactic-co-glycolic acid, random or block copolymers or combinations or mixtures of any of these, or one or more units of polyaminoacids, glycosaminoglycans, polysaccharides, or proteins.

In certain embodiments, the linker comprises a biodegradable linker. In particular embodiments, the biodegradable linker is selected from:

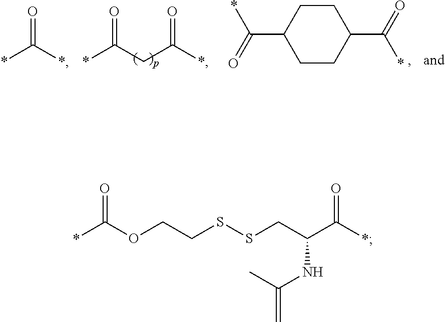

wherein * denotes a point of attachment of the linker to the therapeutic agent and the lysine moiety and p is an integer selected from 1, 2, 3, 4, 5, 6, 7, 8, 9, 10, 11, and 12. In particular embodiments, the linker comprises a succinate moiety having a chemical structure of:

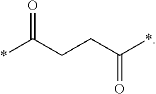

In certain embodiments, the composition of formula (I) comprises:

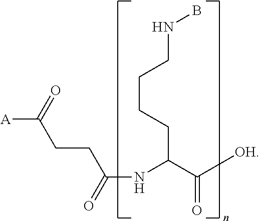

In certain embodiments, the therapeutic agent comprises a hydrophobic or a hydrophilic drug. In more certain embodiments, the drug is selected from an anti-cancer drug, an antibiotic, an antiviral agent, a hemostatic agent, and an anesthetic. In particular embodiments, the antibiotic is selected from a penicillin, a macrolide, a cephalosporin, a fluoroquinolone, a beta-lactam, a tetracycline, trimethoprim-sulfamethoxazole, a urinary anti-infective, a lincosamide, and combinations thereof. In more particular embodiments, the antibiotic is selected from penicillin, amoxicillin, azithromycin, erythromycin, cephalexin, cefdinir, ciprofloxacin, levofloxacin, tetracycline, doxycycline, nitrofurantoin, and clindamycin. In particular embodiments, the anesthetic is selected from bupivacaine, lidocaine, proparacaine, tetracaine, dibucaine, benoxinate, ropivacaine, articaine, carbocaine, marcaine, mepivacaine, polocaine, prilocaine, sensorcaine, and septocaine. In particular embodiments, the anti-cancer drug is selected from camptothecin (CPT), paclitaxel, docetaxel, tamoxifen, and analogues and combinations thereof. In more particular embodiments, the anti-cancer drug comprises camptothecin or an analogue thereof. In certain embodiments, the analogue of camptothecin is selected from topotecan, irinotecan (CPT-11), silatecan (DB-67, AR-67), cositecan (BNP-1350), exatecan, lurtotecan, gimatecan (ST1481), belotecan (CKD-602), and rubitecan.

In certain embodiments, the composition has a chemical structure selected from:

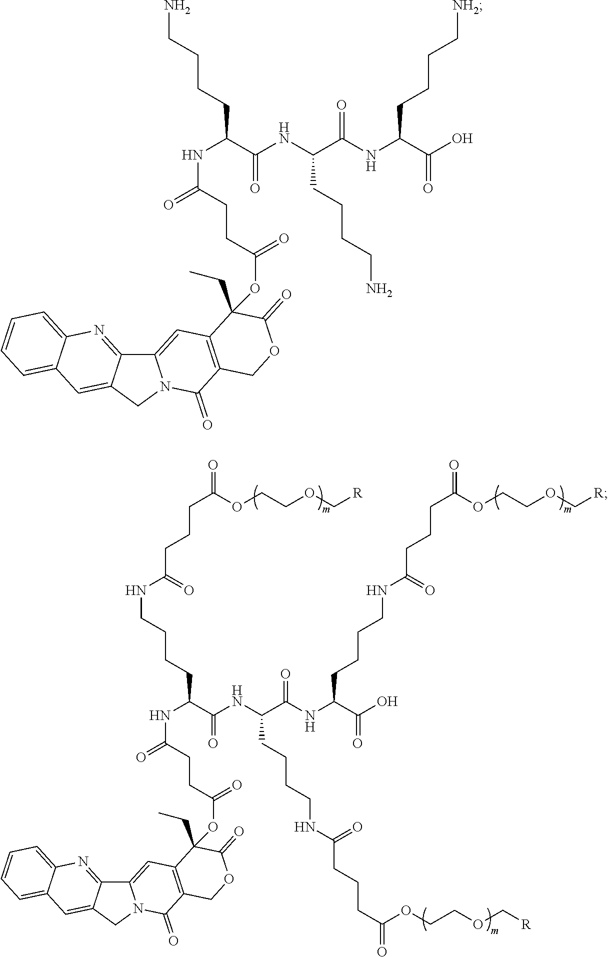

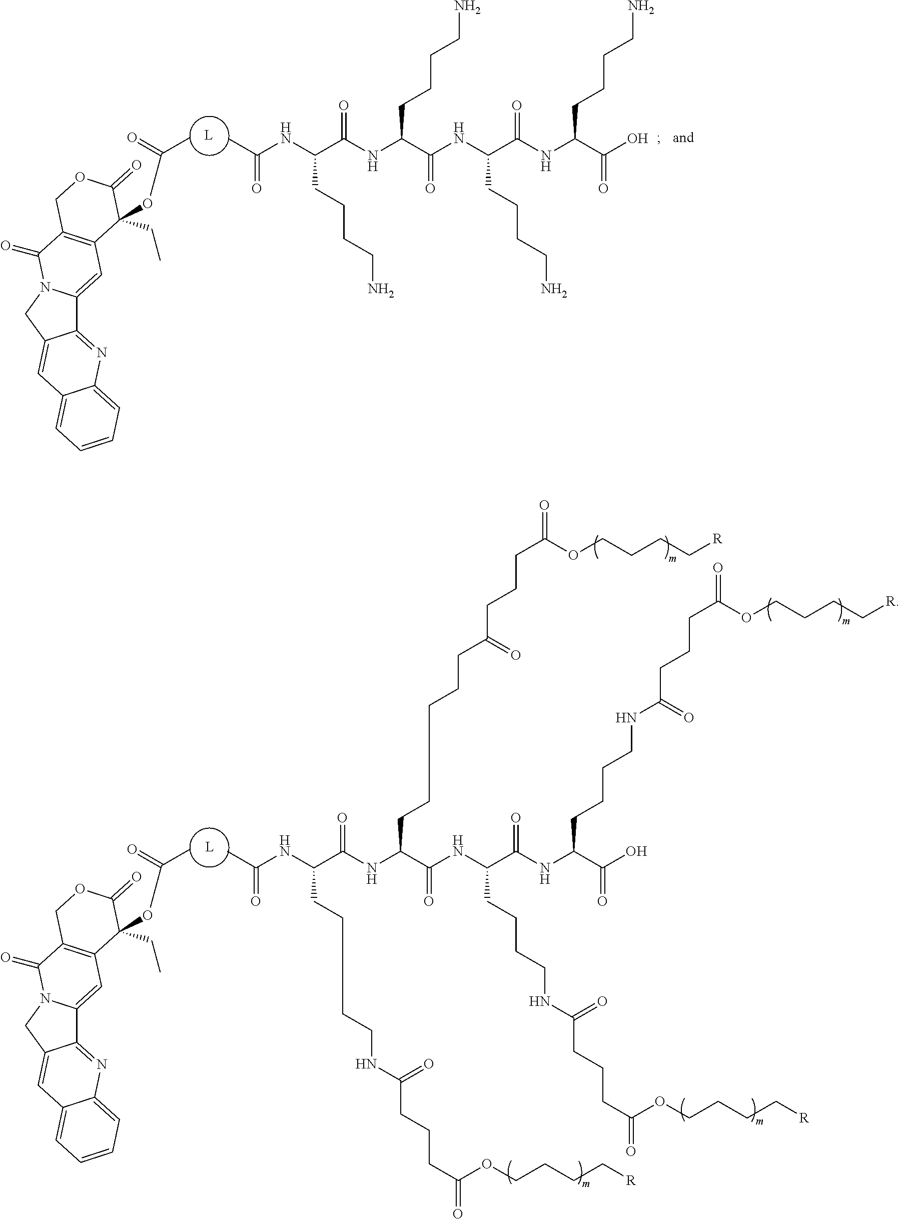

In certain embodiments, the composition of formula (I) has a chemical structure selected from:

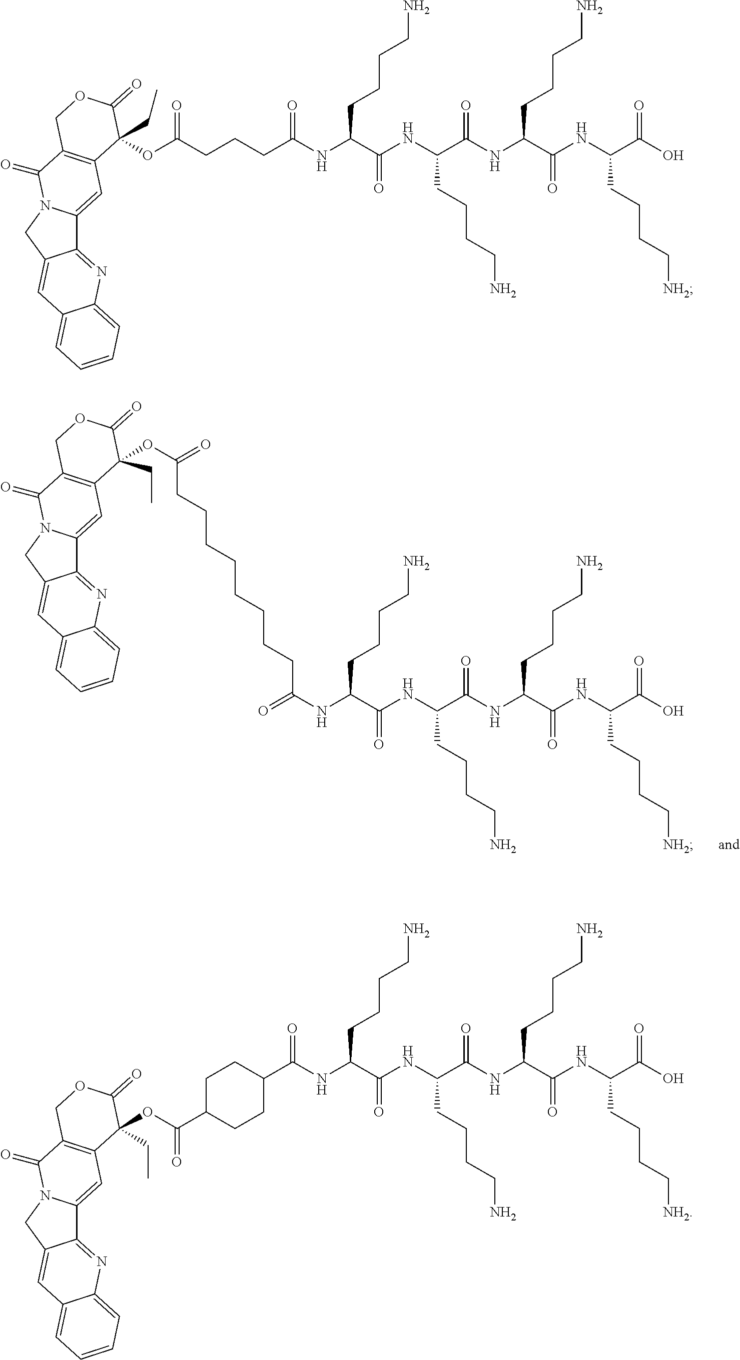 [SMILES: CC[C@]1(OC(=O)CCCC(=O)N[C@@H](CCCCN)C(=O)N[C@@H](CCCCN)C(=O)N[C@@H](CCCCN)C(=O)N[C@@H](CCCCN)C(=O)O)C(=O)OCc2c1cc1n(c2=O)Cc2cc3ccccc3nc2-1 ; CC[C@]1(OC(=O)CCCCCCCCC(=O)N[C@@H](CCCCN)C(=O)N[C@@H](CCCCN)C(=O)N[C@@H](CCCCN)C(=O)N[C@@H](CCCCN)C(=O)O)C(=O)OCc2c1cc1n(c2=O)Cc2cc3ccccc3nc2-1 ; CC[C@@]1(OC(=O)C2CCC(C(=O)N[C@@H](CCCCN)C(=O)N[C@@H](CCCCN)C(=O)N[C@@H](CCCCN)C(=O)N[C@@H](CCCCN)C(=O)O)CC2)C(=O)OCc2c1cc1n(c2=O)Cc2cc3ccccc3nc2-1]

In certain embodiments of the compounds of formula (I) provided herein, the amine group, e.g., —NH₂, can be replaced by PEG group, including a multi-arm PEG group.

Throughout the specification and claims, a given chemical formula or name shall encompass all tautomers, congeners, and optical- and stereoisomers, as well as racemic mixtures where such isomers and mixtures exist.

In certain embodiments, the presently disclosed composition further comprises a buffer. In particular embodiments, the buffer comprises a borate buffer. In more particular embodiments, the borate buffer comprises a nano-confined borate buffer.

In certain embodiments, the composition exhibits one or more properties selected from: (a) solubility in a borate buffer; (b) stability in a borate buffer; and (c) does not assemble into a nanostructure in either PBS or water.

In other embodiments, the formulation comprises: (a) a first precursor solution comprising a composition described herein, e.g., a compound of formula (I) comprising one or more terminal amine groups; and (b) a second precursor comprising polyethylene glycol, e.g., PEG moiety as described herein. In certain embodiments, the first precursor solution comprises a borate buffer. In particular embodiments, the borate buffer has a concentration ranging from 2 mM to about 100 mM. In more particular embodiments, the borate buffer in confined in a nanoparticle.

In other embodiments, the presently disclosed subject matter provides a method for treating a disease, disorder, or condition in a subject, the method comprising administering a composition described herein to a subject in need of treatment thereof. In certain embodiments, the disease, disorder, or condition is selected from a cancer, an infection, and an inflammation. In particular embodiments, the cancer is selected from breast cancer, ovarian cancer, colon cancer, stomach cancer, non-small cell lung cancer (NSCLC), a glioblastoma, and Kaposi sarcoma.

The drug-loaded hydrogel allows for the release of therapeutic molecules by hydrolysis of the ester linker in vivo. This biocompatible hydrogel is absorbed in a time frame of about 4 weeks, which is sufficient for wound healing and sustained drug delivery.

In other embodiments, the presently disclosed subject matter provides a composition described herein and instructions for use. In certain embodiments, the kit further comprises (a) a first precursor solution comprising a composition described herein; and (b) a second precursor comprising polyethylene glycol. In particular embodiments, the kit further comprises one or more components selected from one or more solvent or buffers, one or more vials, one or more syringes, and instructions for use. Upon syringe- or spray-administration through an applicator kit, the cross-linked hydrogels are formed in situ within seconds.

In other embodiments, the presently disclosed subject matter provides a sealant comprising the composition described herein.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In other embodiments, the presently disclosed subject matter provides a kit comprising a presently disclosed composition and instructions for use. In yet other embodiments, the kit further comprises: (a) a first precursor solution comprising a the presently disclosed composition; and (b) a second precursor comprising polyethylene glycol. In certain embodiments, the kit further comprises one or more components selected from one or more solvent or buffers, one or more vials, one or more syringes, and instructions for use.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments 0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Preparation, Characterization, and Properties of the Presently Disclosed Compositions

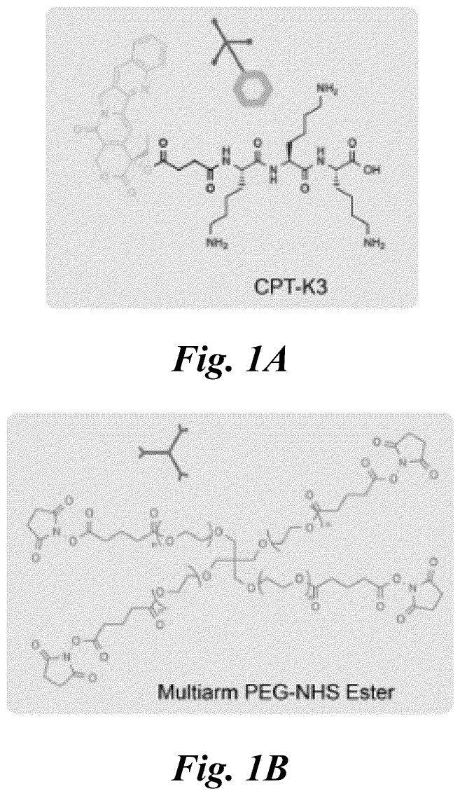

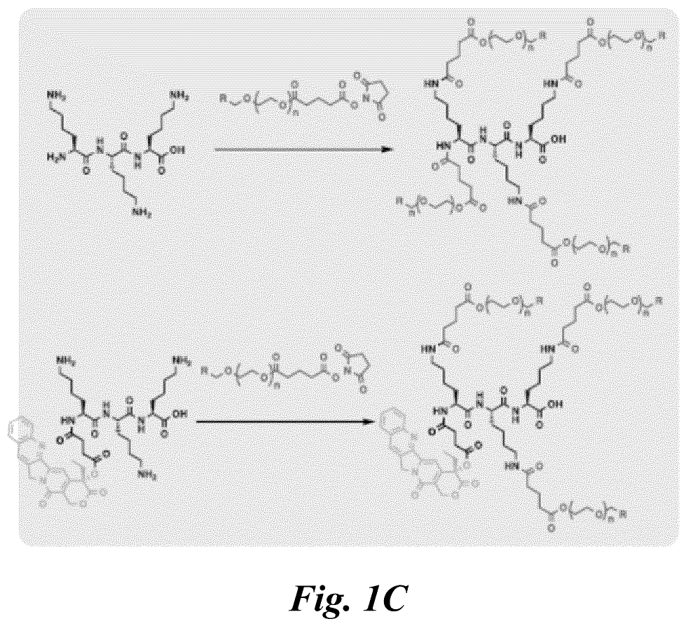

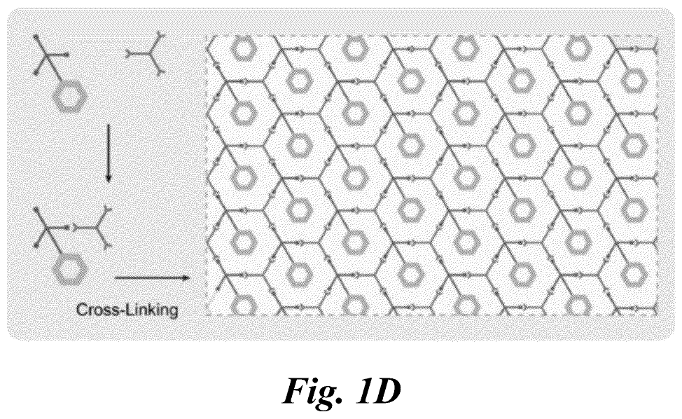

Referring now to FIG. 1, is a schematic illustrating a representative method for preparing the presently disclosed sealant system incorporating, for example, the small molecular hydrophobic anticancer drug camptothecin (“CPT”) as a model drug. FIG. 1A is the chemical structure of camptothecin linked to trilysine (“K3”) (referred to herein as “CPT-K3”) through a succinate linking moiety. FIG. 1B is the chemical structure of a multi-arm PEG-NHS ester. FIG. 1C is a representative method for preparing the presently disclosed sealant system incorporating the small molecular hydrophobic anticancer drug camptothecin (CPT) as a model drug in which CPT-K3 is conjugated with a multi-arm PEG-NHS ester. FIG. 1D is schematic showing a representative cross-linked system.

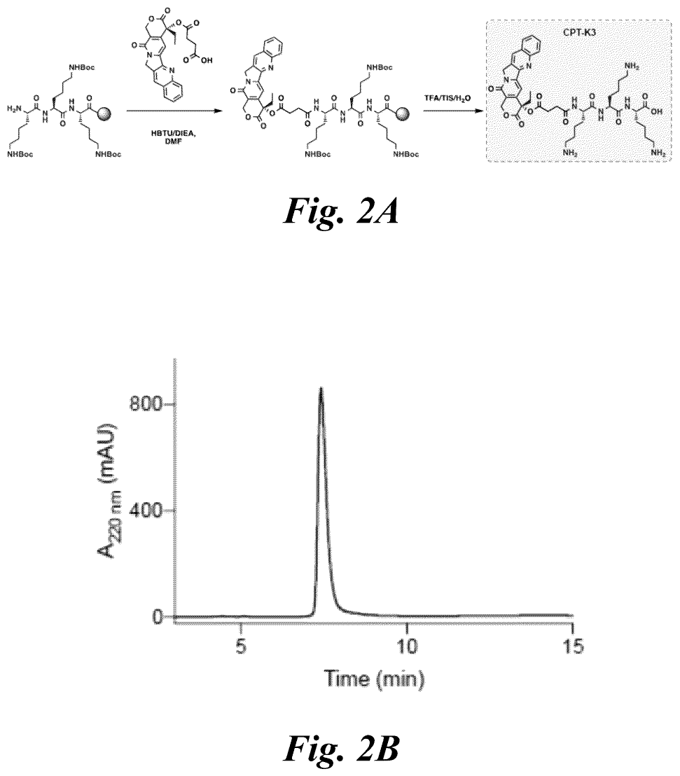

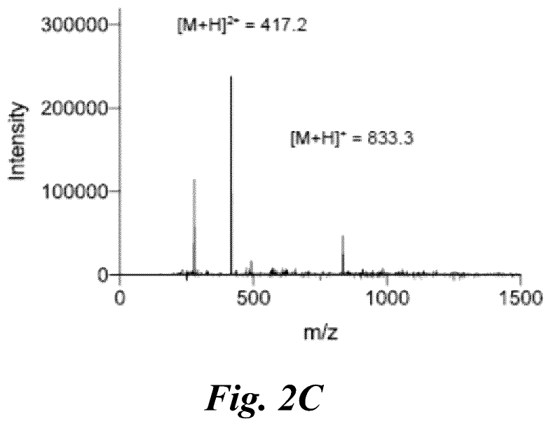

Referring now to FIG. 2A is a reaction scheme showing the covalent linkage of CPT to the N-terminus of trilysine via a succinate linker to form the CPT-K3 conjugate through solid-phase peptide synthesis (SPSS). The successful synthesis was confirmed by HPLC (FIG. 2B) and ESI-MS (FIG. 2C).

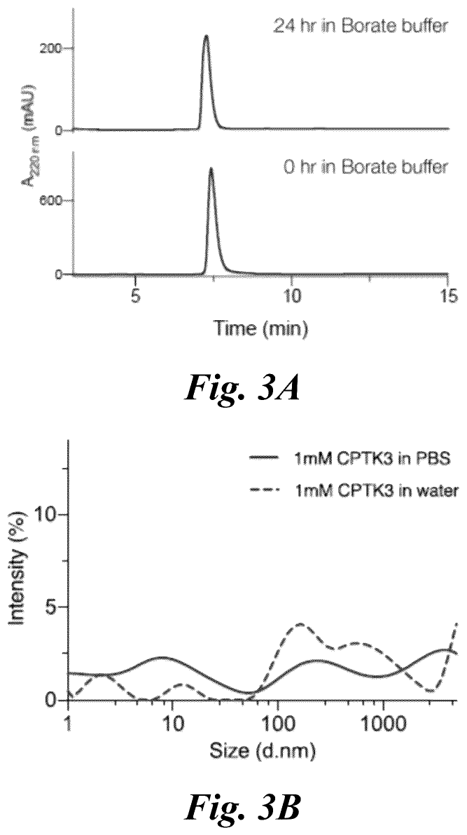

Referring now to FIG. 3, CPT-K3 remains stable in a clear precursor solution. FIG. 3A shows that CPT-K3 exhibits good solubility in borate buffer and is stable in 75-mM borate buffer for at least 24 hours and FIG. 3B indicates that CPT-K3 did not assemble into a nanostructure in either PBS or water.

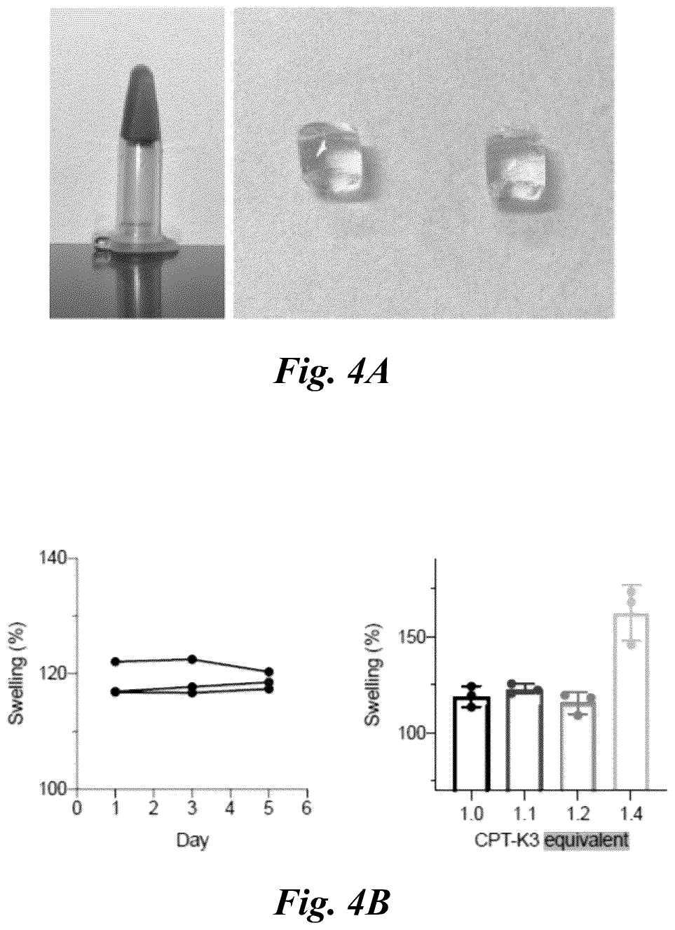

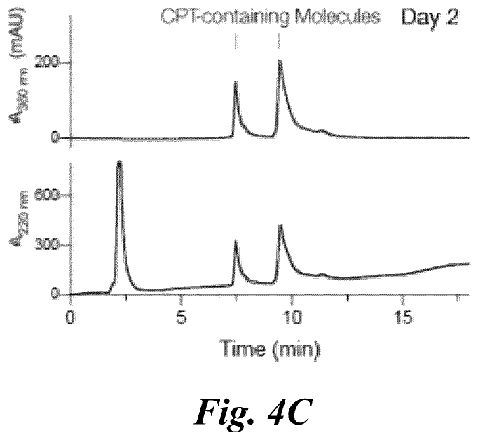

Referring now to FIG. 4A, FIG. 4B, and FIG. 4C, FIG. 4A shows photographs showing that CPT-K3 forms a hydrogel after crosslinking in a 1:1 stoichiometry; FIG. 4B shows an average swelling rate of 120% in 1.0 to 1.2 CPT-K3 equivalent; and FIG. 4C shows CPT release monitored by HPLC.

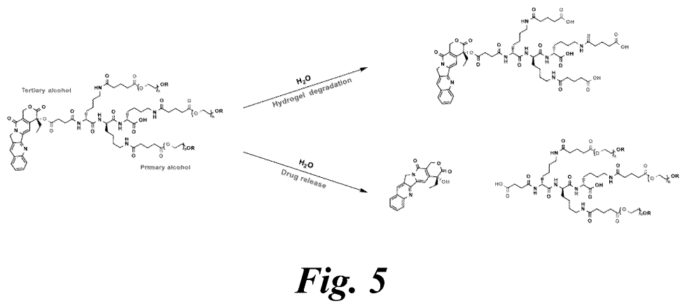

FIG. 5 demonstrates the release of CPT by ester hydrolysis. Shown is hydrogel degradation yielding a conjugate comprising a product having primary alcohol groups on the residual PEG moieties. Also shown in FIG. 5 is release of the therapeutic agent, e.g., a drug, yielding the drug and the remaining linker, e.g., a succinate, and PEGylated trilysine, wherein the terminal end of the succinate linker comprises a tertiary alcohol.

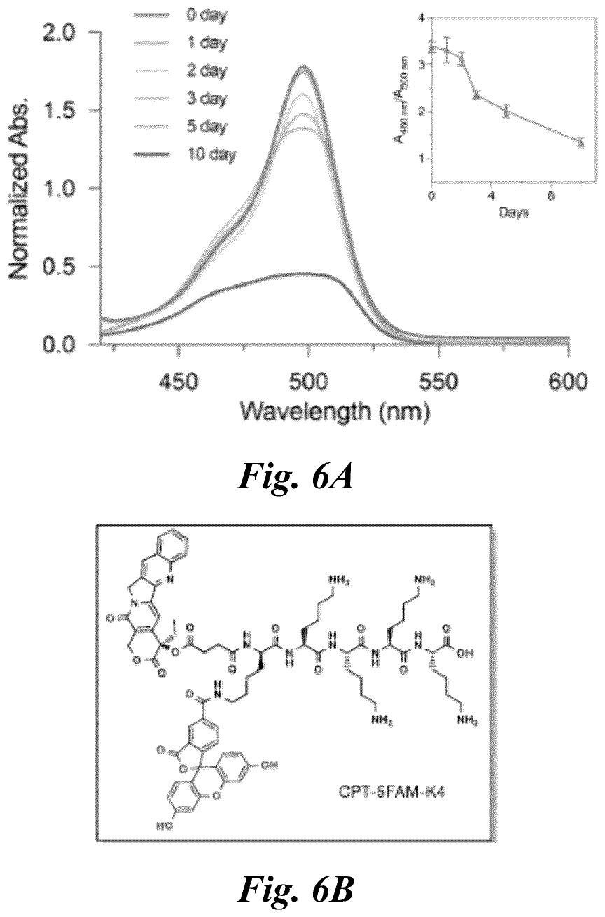

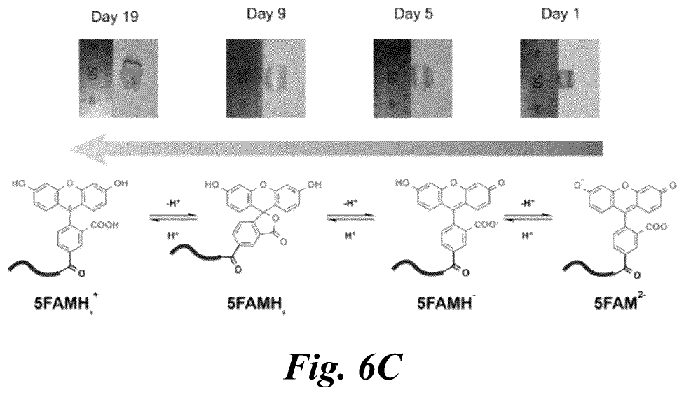

FIG. 6A, FIG. 6B, and FIG. 6C show that a color shift in 5-carboxyfluorescein (5-FAM) hydrogel indicated a gradual decrease in local pH over hydrogel degradation. FIG. 6A shows the normalized absorbance at day 0, day 1, day 2, day 3, day 5, and day 10, whereas the inset shows the ratio of A_{460 nm}/A_{500 nm} from 0 days to 10 days; FIG. 6B is the chemical structure of CPT-5FAM-K4; FIG. 6C shows photographs at day 1, day 5, day 9, and day 19 of the 5-FAM and structures of the 5-FAM dye over time during degradation and change in local pH.

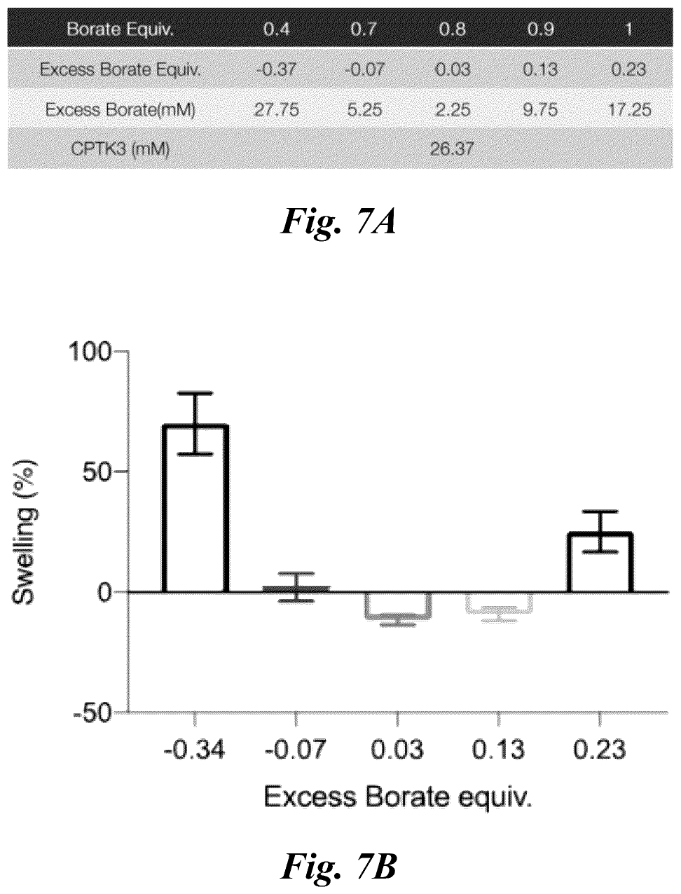

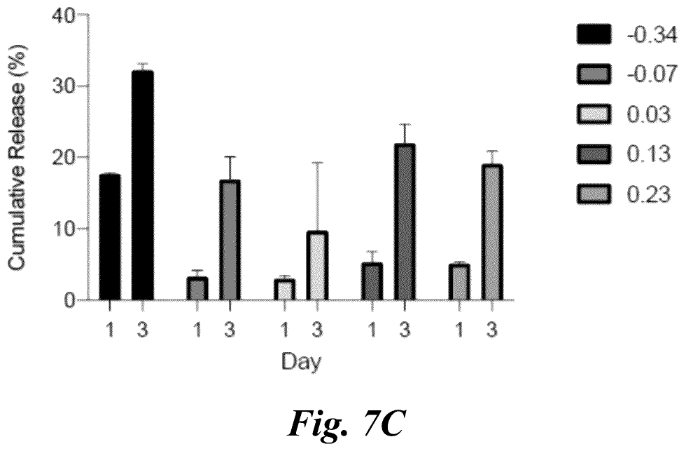

FIG. 7A, FIG. 7B, and FIG. 7C demonstrate CPTK3-8A release with different borate concentrations. FIG. 7A is a table providing the borate equivalent, excess borate equivalent, excess borate (mM), and CPTK3 (mM). FIG. 7B is a plot of the swelling (%) vs. excess borate equivalent. FIG. 7C is a plot of the cumulative release (%) per day of excess borate equivalent. In this example, the hydrogel release of CPTK3-8A was repeated. Note that CPT was found to precipitate onto hydrogel surfaces in the last run.

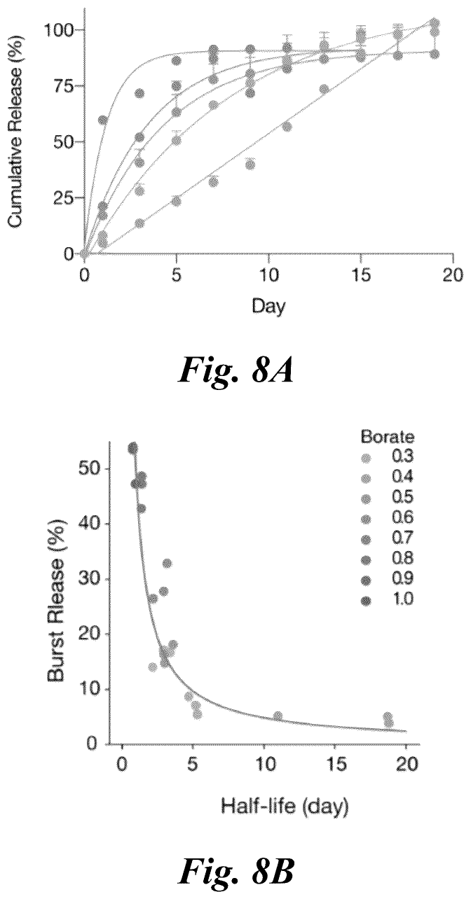

FIG. 8A and FIG. 8B demonstrate that drug release in CPTK4/A4 hydrogel is controlled by nano-confined borate. FIG. 8A is a plot of the cumulative release (%) vs. day. FIG. 8B is a plot of the burst release (%) vs. half-life (day). Each graph depicts borate at 0.3, 0.4, 0.5, 0.6, 0.7, 0.8, 0.9, and 1.0.

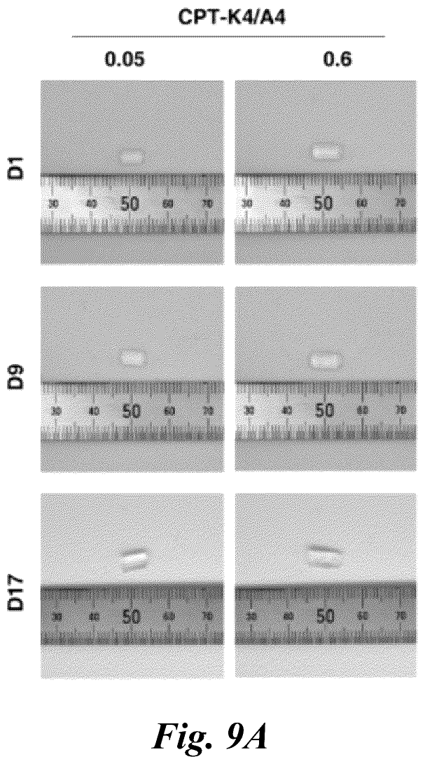

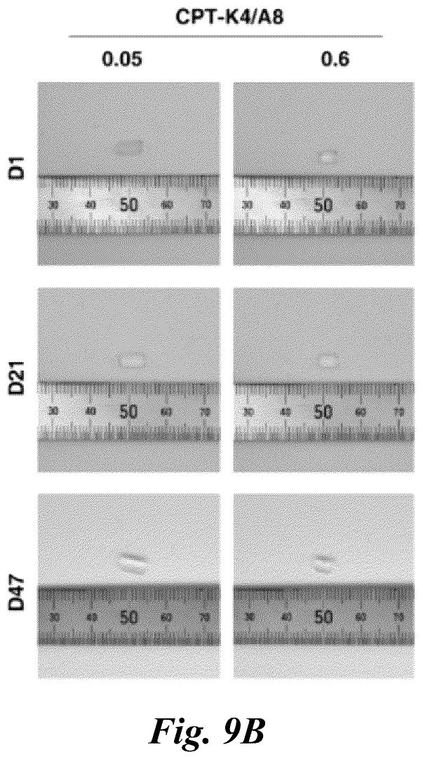

FIG. 9 demonstrates that nano-confined borate controls hydrogel degradation. Left panel is CPT-K4/A4. Right panel is CPT-K4/A8.

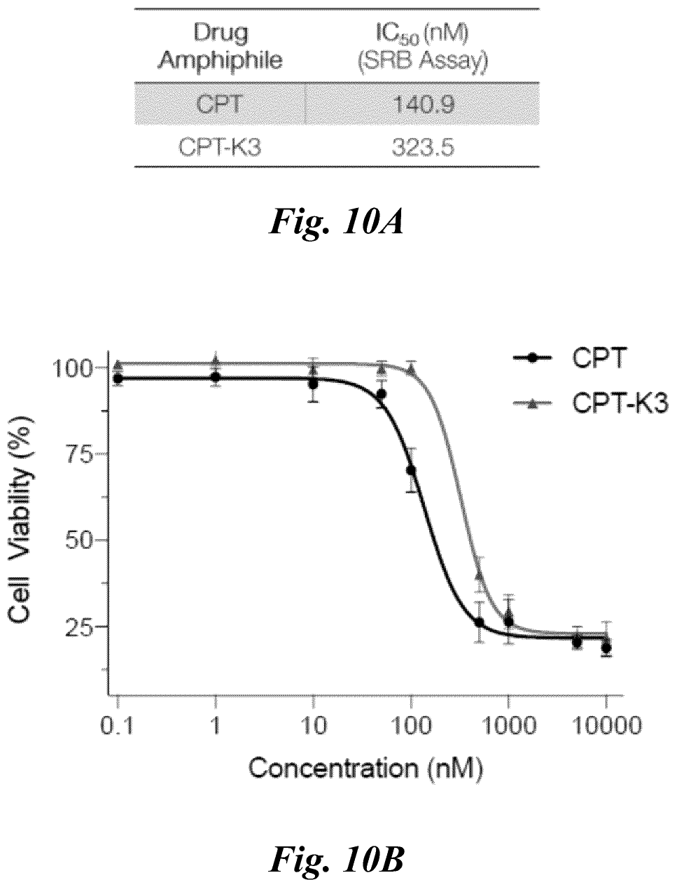

FIG. 10A and FIG. 10B demonstrate that CPT-K3 exhibited in vitro cytotoxicity against U87 glioblastoma cells. FIG. 10A is a table providing the IC50 of CPT and CPT-K3. FIG. 10B is a plot of cell viability (%) vs. concentration. In this example, cell viability (%) was determined using the SRB method. CPT and CPT-K3 exhibited comparable cytotoxicity against the U87 glioblastoma cell line.

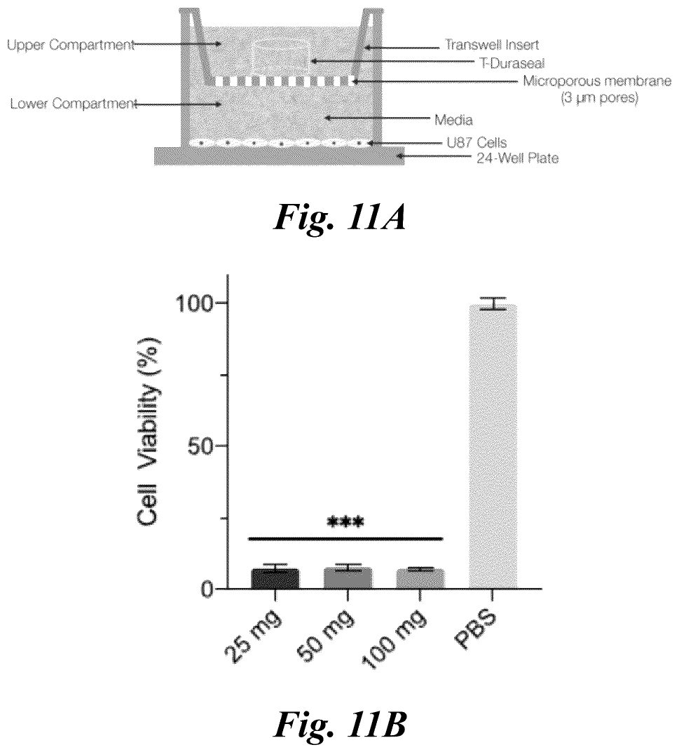

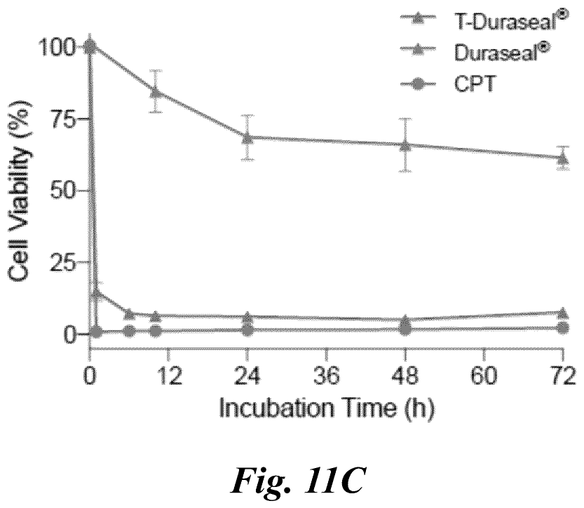

FIG. 11A, FIG. 11B, and FIG. 11C demonstrate the in vitro cytotoxicity of the presently disclosed sealant system against U87 cells. FIG. 11A is a schematic showing a representative experimental setup for measuring the cytotoxicity of the presently disclosed sealant system. FIG. 11B is a plot of the cell viability (%) vs. dose of the presently disclosed sealant system, including 25 mg, 50 mg, 100 mg, and PBS. FIG. 11C is a plot of the cell viability vs. incubation time for two hydrogel-based compositions known in the art and CPT. In this example, the cytotoxicity of the presently disclosed sealant system was directly assessed using Transwell. The example demonstrates that the cell viability between 25 mg, 50 mg, and 100 mg the presently disclosed sealant system were not significant.

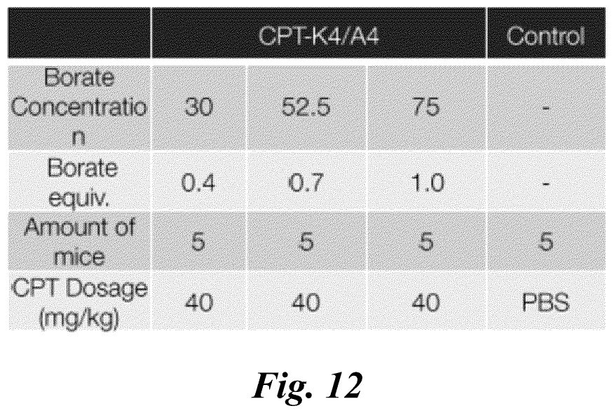

FIG. 12 shows the in vivo therapeutic effect of CPT-K4/A4 against U87 tumor. In this example, the tumor inhibition rate was evaluated after treatments. The procedure was as follows: inoculate nude mice (6-7 weeks, 15 g-25 g) with the U87 human glioblastoma tumor cell line subcutaneously. Then, wait until the tumor volume reaches to 100 mm³. Then inject the four groups of formulations peritumorally. Each group includes five mice with 35 nude mice in total. The dosage was 40 mg/kg equivalent. 0.7 mL hydrogel (0.35 mL+0.35 mL).

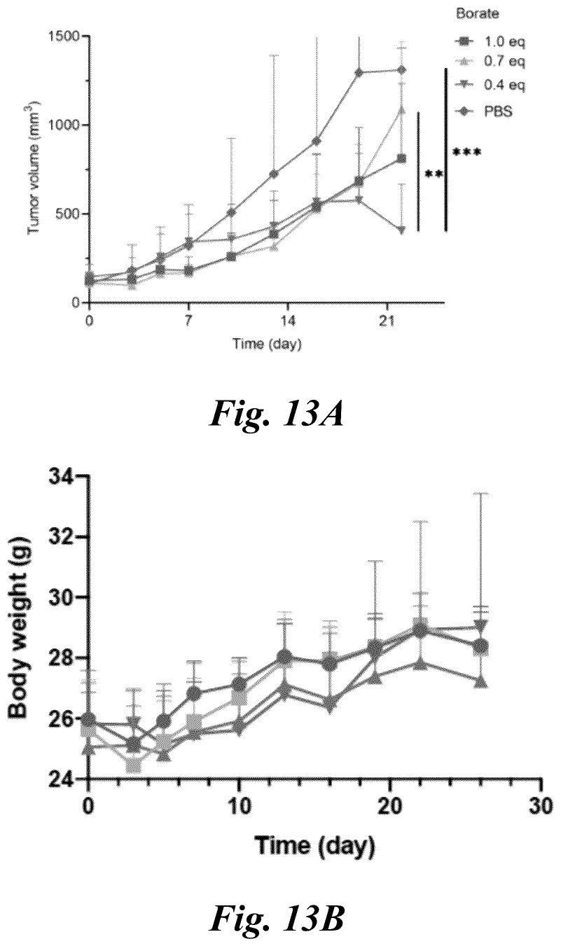

FIG. 13A and FIG. 13B demonstrate the in vivo therapeutic effect against U87 tumor. FIG. 13A and FIG. 13B demonstrate the in vivo therapeutic effect against U87 tumor. FIG. 13A is a plot of the tumor volume (mm³) vs. time (day). FIG. 13B is a plot of body weight (g) vs. time (day).

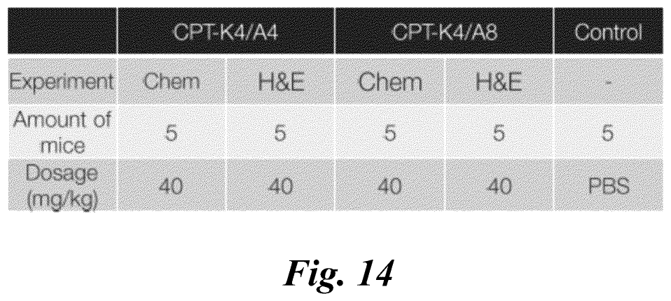

FIG. 14 shows the safety and biocompatibility of the presently disclosed sealant system. In this example, 100 total C57BL/6 mice were used. With regard to serum biochemistry and whole blood cell count, the following procedure was used. Inject the five groups of formulations (100 μg/25 μL×2, CPT equivalent) subcutaneously on the back of the C57BL/6 mice (6-7 weeks, 15 g-25 g). Each group will include five mice. Then, analyze whole blood cell count, serum biochemistry (ALP, ALT, AST, BUN, CREA, and TP). Collect blood at day 7 and day 14 after sacrificing the mice. 25 C57BL/6 mice total.

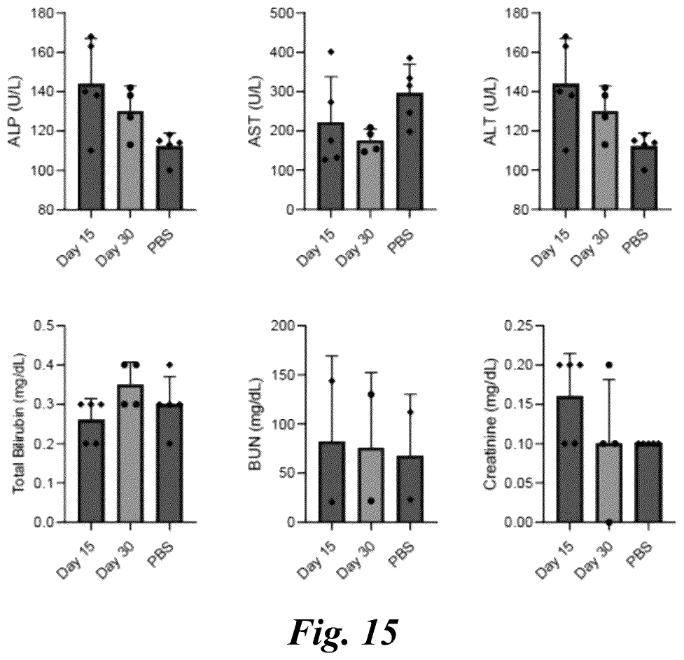

FIG. 15 shows the serum chemistry of CPT-K4/4A the presently disclosed sealant system. Shown are plots of the ALP (U/L), AST (U/L), ALT (U/L), total bilirubin (mg/dL), BUN (mg/dL), and creatinine (mg/dL) at day 15 and day 30 compared to PBS.

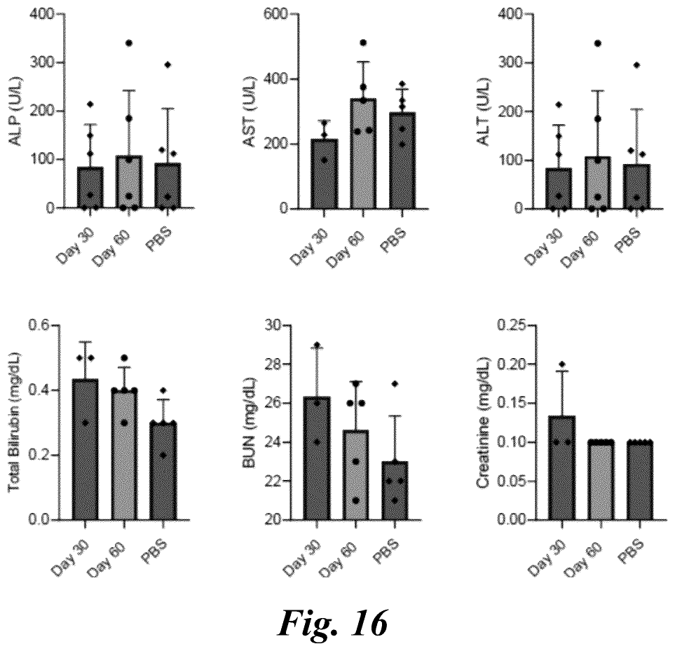

FIG. 16 shows the serum chemistry of CPT-K4/8A. Shown are plots of the ALP (U/L), AST (U/L), ALT (U/L), total bilirubin (mg/dL), BUN (mg/dL), and creatinine (mg/dL) at day 15 and day 30 compared to PBS.

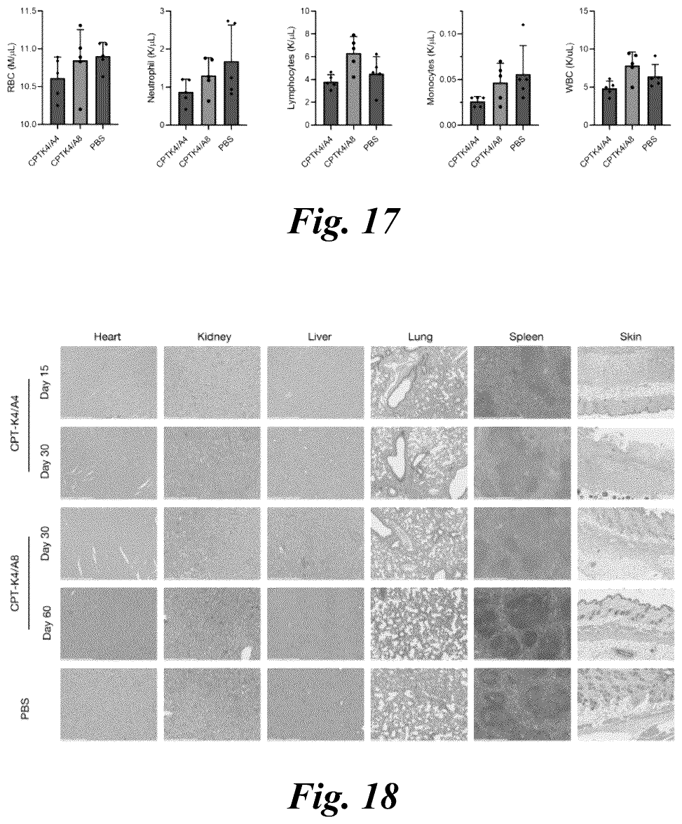

FIG. 17 shows the total blood cell count. Shown are plots of RBC (M/μL), neutrophil (K/μL), lymphocytes (K/μL), monocytes (K/μL), and WBC (K/μL) for CPTK4/A4, CPTK$/A8, and PBS. CPTK4/A4 @ day 14. CPTK4/A8 @ day 60.

FIG. 18 shows H&E-stained tissue sections of heart, kidney, liver, lung, spleen, and skin for CPT-K4/A4 at day 15 and day 30 and CPT-K4/A8 at day 30 and day 60 compared to PBS.

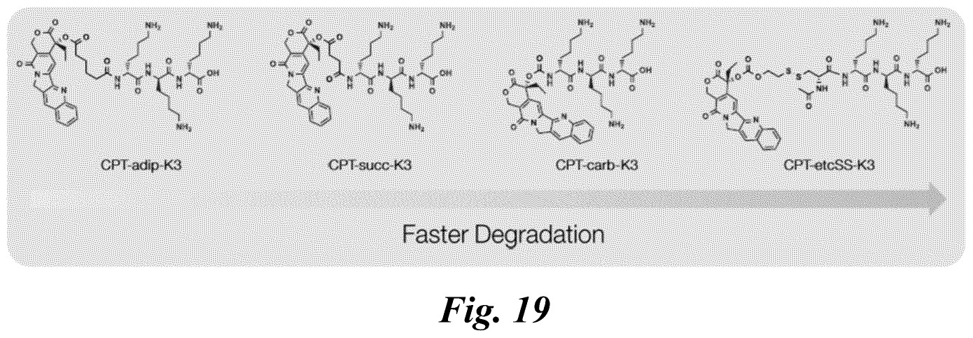

FIG. 19 shows illustrative examples for optimizing drug release with linker design. Without wishing to be bound to any one particular theory, it is thought that the linker group of the presently disclosed drug conjugates has a significant effect on the drug release efficiency and their consequent therapeutic efficacy.

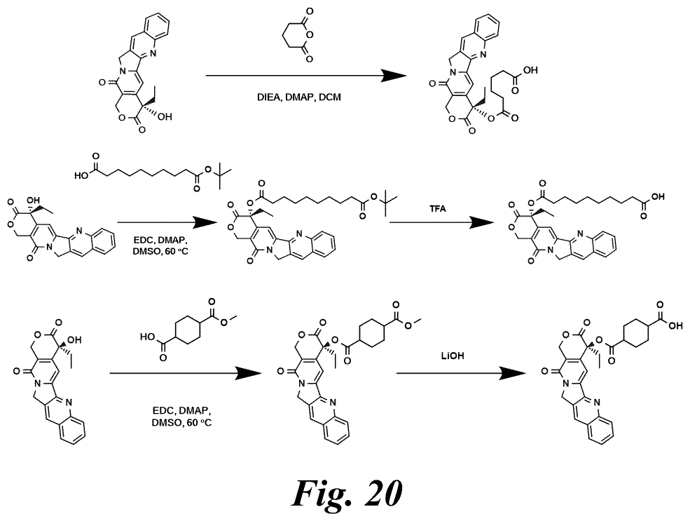

FIG. 20 shows the synthesis schemes for representative camptothecin (CPT) prodrugs.

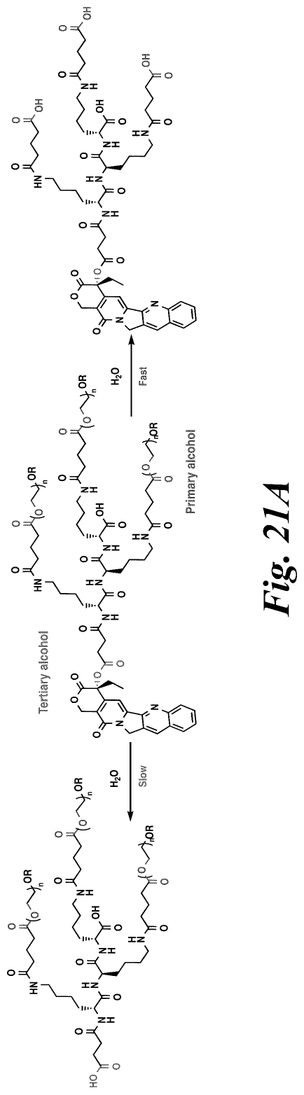

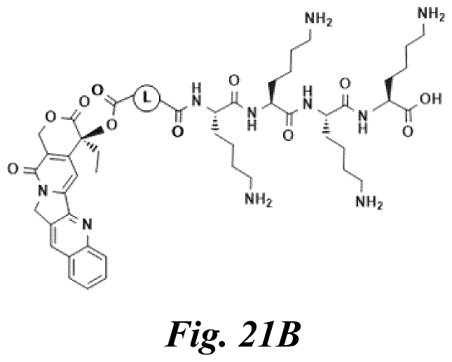

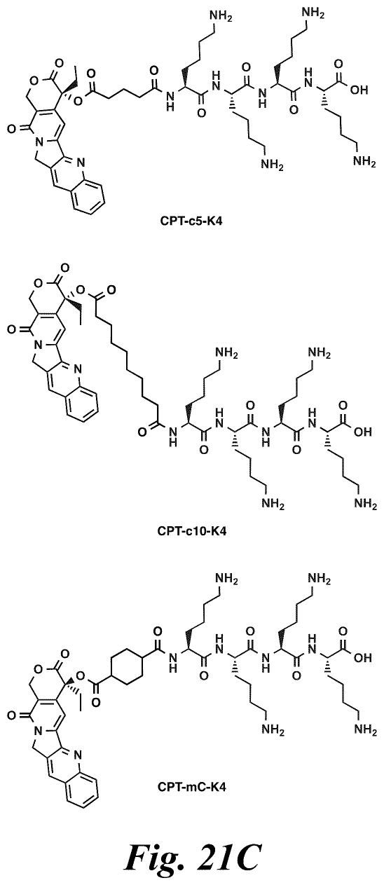

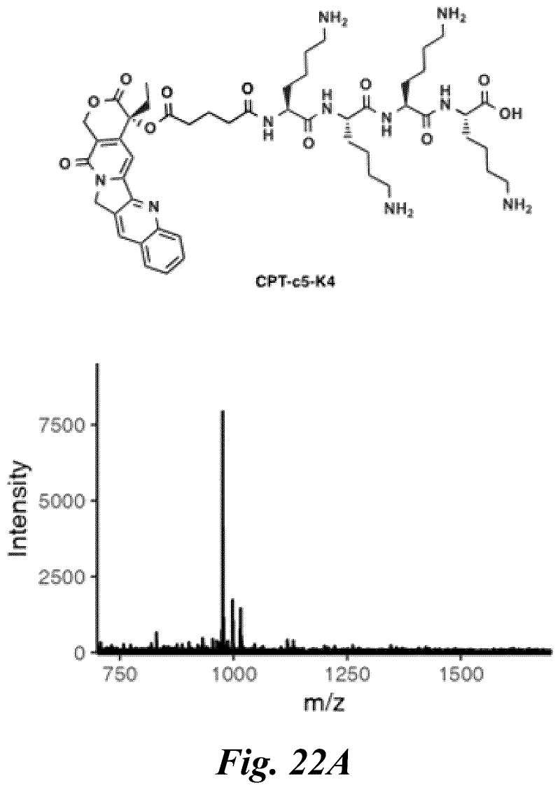

FIG. 21A demonstrates release of CPT in vitro. FIG. 21B shows a representative generic structure of the presently disclosed sealant system with L in this embodiment representing a hydrophobic linker. FIG. 21C shows representative compounds having a hydrophobic linker, e.g., CPT-c5-K4 with a five carbon (c5) linker, CPT-c10-K4 with a ten carbon (c10) linker; and CPT-mC-K4 with a cyclohexylene linker.

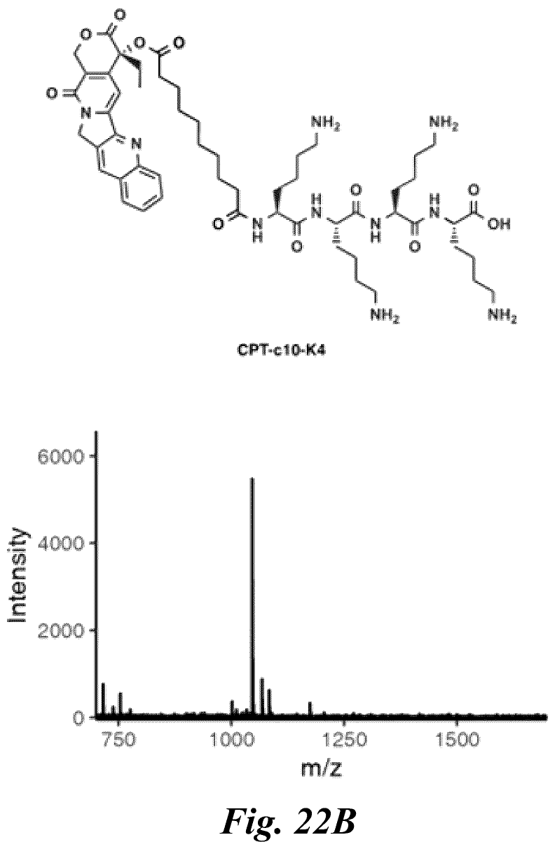

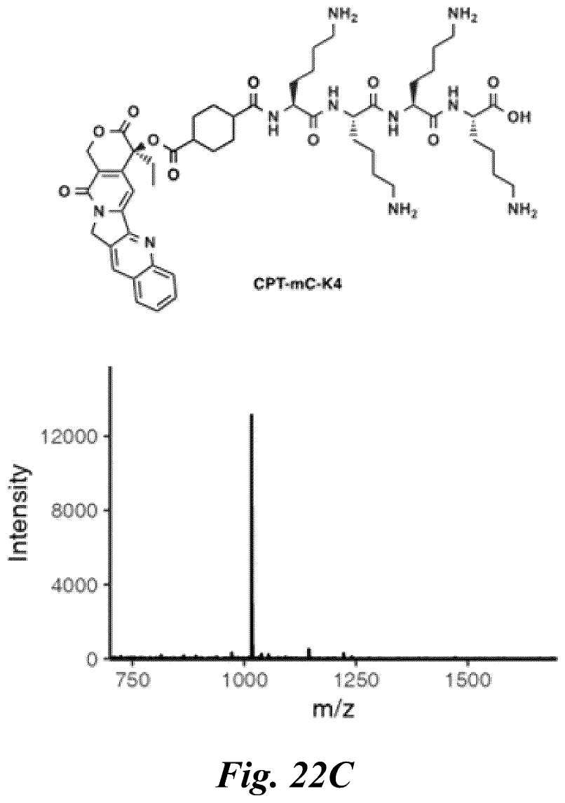

FIG. 22A, FIG. 22B, and FIG. 22C show MALDI-Tof MS of (FIG. 22A) CPT-c5-K4; (FIG. 22B) CPT-c10-K4; and (FIG. 22C) CPT-mC-K4.

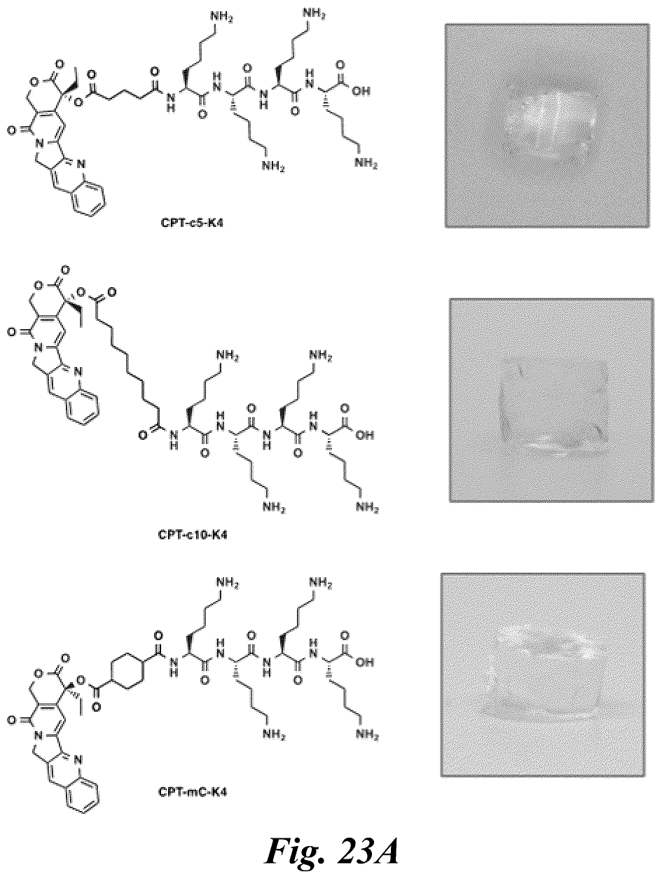

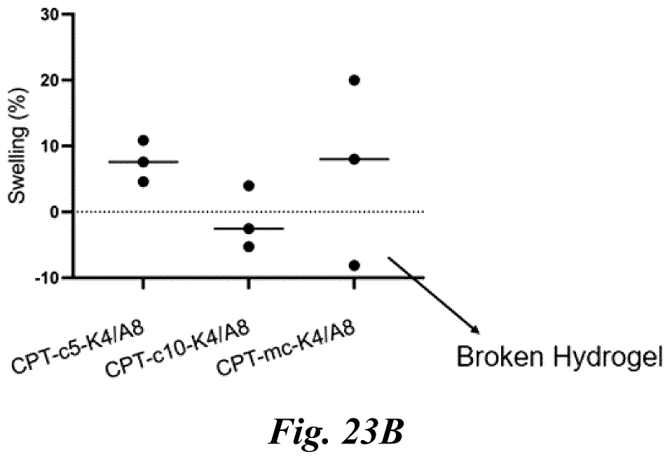

FIG. 23A and FIG. 23B show hydrogel formation of CPT-L-K4 with 20k 8A PEG NHS, for various linkers, L, including linkers referred to herein as c5, c10, and mC. FIG. 23A shows photographs demonstrating that CPT-mC-K4/8A formed brittle hydrogels, possibly due to the rigid mC linker. FIG. 23B is a graph demonstrating that low swelling ratio (<10) were observed in all hydrogels.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
U.S. Pat. No. 6,566,406 for Biocompatible Crosslinked Polymers to Henise et al., issued May 20, 2003, expired.
U.S. Pat. No. 7,009,034 for Biocompatible Crosslinked Polymers to Pathak et al., issued Mar. 7, 2006, expired.
U.S. Pat. No. 7,332,566 for Biocompatible Crosslinked Polymers with Visualization Agents, to Pathak et al., issued Feb. 19, 2008, expired.
U.S. Pat. No. 7,592,418 for Biocompatible Crosslinked Polymers with Visualization Agents, to Pathak et al., issued Sep. 22, 2009, expired.
U.S. Pat. No. 8,003,705 for Biocompatible Hydrogels Made with Small Molecule Precursors, to Sawhney, issued Aug. 23, 2011, expired.
U.S. Pat. No. 8,535,705 for Biocompatible polymers and hydrogels and methods of use, to Pathak et al., issued Sep. 17, 2013.
U.S. Patent Application Publication No. 20150352246 for Sealants Having Controlled Degration, to Henise et al., published Dec. 10, 2015, abandoned Jan. 21, 2020.
U.S. Patent Application Publication No. 20090227689 for Low-Swelling Biocompatible Hydrogels, to Bennett, published Sep. 10, 2009, abandoned Jan. 23, 2012.
International PCT Patent Application Publication No. WO2022103944 for Hydrogels Formed In Situ and Composition Design for Intrauterine Use, to Bassett and Feldberg, published May 19, 2022.
International PCT Patent Application Publication No. WO2022066891 for Sustained Release Biodegradable Intracanalicular Inserts Comprising a Hydrogel and an Active Agent, to Blizzard et al., published May 31, 2022.
International PCT Patent Application Publication No. WO2022066884 for Sustained Release Biodegradable Intracanalicular Inserts Comprising a Hydrogel and Cyclosporine, to Blizzard et al., published May 31, 2022.
International PCT Patent Application Publication No. WO2020219823 for Intracanalicular Hydrogel Inserts for the Delivery of Anesthetics, to Jarrett et al., published Oct. 29, 2020.

Although the foregoing subject matter has been described in some detail by way of illustration and example for purposes of clarity of understanding, it will be understood by those skilled in the art that certain changes and modifications can be practiced within the scope of the appended claims.

1. A composition of formula (I): 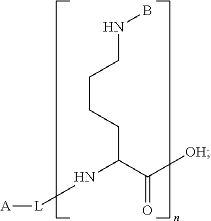
wherein:
n is an integer selected from 2, 3, 4, and 5;
A is a therapeutic agent;
L is a linker; and
each B is independently a multi-arm polyethylene glycol (PEG) moiety, wherein each B can be the same or different.

2. The composition of claim 1, wherein the composition of formula (I) is selected from:

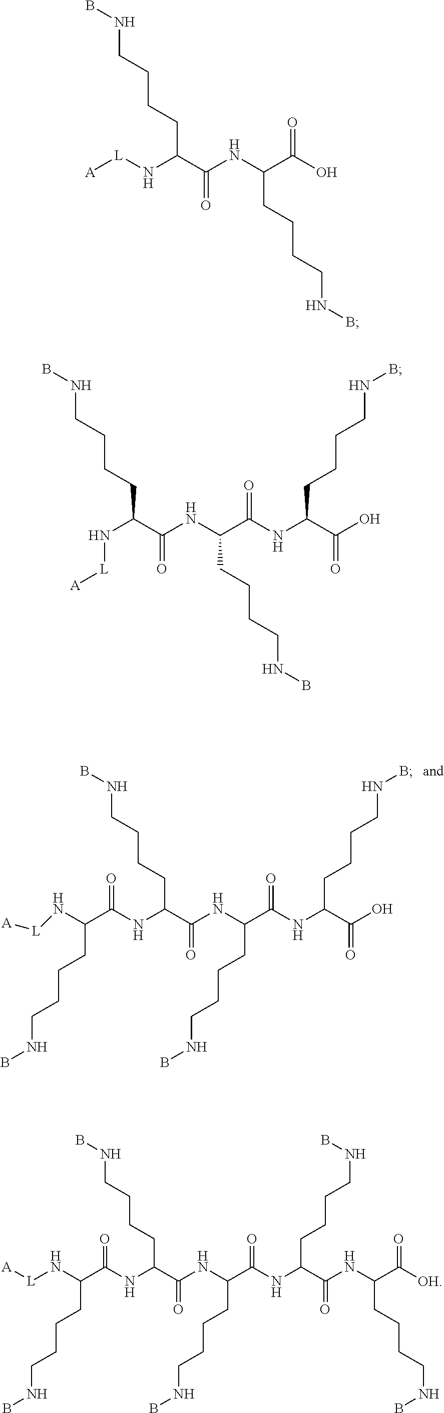

3. The composition of claim 1, wherein the composition of formula (I) comprises:

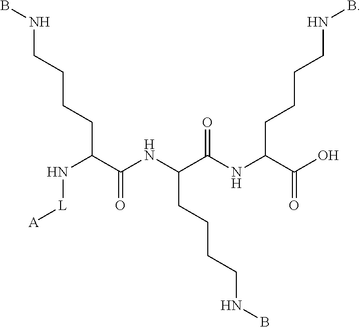

4. The composition of claim 1, wherein the composition of formula (I) comprises: 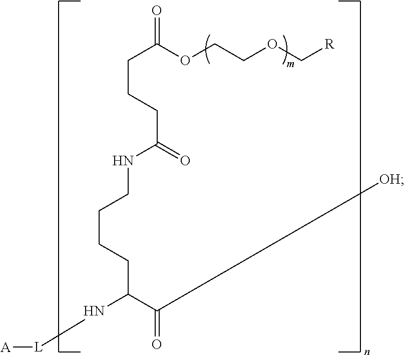
wherein:
each m is independently an integer selected from 1 to 1,000;
each n is independently an integer selected from 1 to 2,000; and
each R independently comprises a core of a multi-arm PEG moiety.

5. The composition of claim 4, wherein the composition of formula (I) comprises:

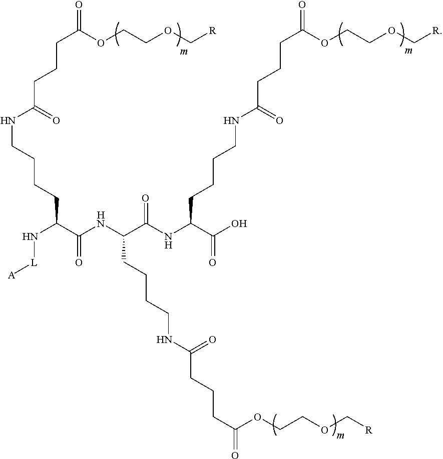

6. The composition of claim 1, wherein the multi-arm PEG moiety has between 2 and 10 arms.

7. The composition of claim 6, wherein the multi-arm PEG moiety is selected from a 2-arm PEG moiety, a 4-arm PEG moiety, and an 8-arm PEG moiety.

8. The composition of claim 1, wherein the multi-arm PEG moiety further comprises an end group selected from —OH, —SH, —NH₂, —N₃, —CH═CH₂, —C(CH₃)═CH₂, —C≡CH, —COOH, alkoxyl, halogen, a succinimidyl ester, and a maleimide.

9. The composition of claim 1, wherein the linker comprises a biodegradable linker.

10. The composition of claim 9, wherein the biodegradable linker is selected from: 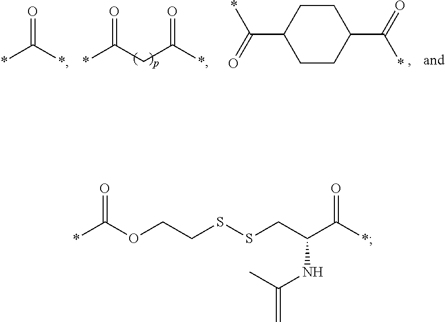
wherein * denotes a point of attachment of the linker to the therapeutic agent and the lysine moiety and p is an integer selected from 1, 2, 3, 4, 5, 6, 7, 8, 9, 10, 11, and 12.

11. The composition of claim 10, wherein the linker comprises a succinate moiety having a chemical structure of:

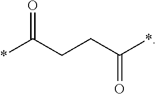

12. The composition of claim 11, wherein the composition of formula (I) comprises:

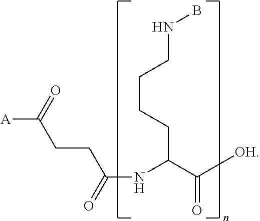

13. The composition of claim 1, wherein the therapeutic agent comprises a hydrophobic or a hydrophilic drug.

14. The composition of claim 13, wherein the drug is selected from an anti-cancer drug, an antibiotic, an antiviral agent, a hemostatic agent, and an anesthetic.

15. The composition of claim 14, wherein:
(a) the antibiotic is selected from a penicillin, a macrolide, a cephalosporin, a fluoroquinolone, a beta-lactam, a tetracycline, trimethoprim-sulfamethoxazole, a urinary anti-infective, a lincosamide, and combinations thereof;
(b) wherein the anesthetic is selected from bupivacaine, lidocaine, proparacaine, tetracaine, dibucaine, benoxinate, ropivacaine, articaine, carbocaine, marcaine, mepivacaine, polocaine, prilocaine, sensorcaine, and septocaine; and/or
(c) the anti-cancer drug is selected from camptothecin (CPT), paclitaxel, docetaxel, tamoxifen, and analogues and combinations thereof.

16. The composition of claim 15, wherein the antibiotic is selected from penicillin, amoxicillin, azithromycin, erythromycin, cephalexin, cefdinir, ciprofloxacin, levofloxacin, tetracycline, doxycycline, nitrofurantoin, and clindamycin.

17-18. (canceled)

19. The composition of claim 15, wherein the anti-cancer drug comprises camptothecin or an analogue thereof.

20. The composition of claim 19, wherein the analogue of camptothecin is selected from topotecan, irinotecan (CPT-11), silatecan (DB-67, AR-67), cositecan (BNP-1350), exatecan, lurtotecan, gimatecan (ST1481), belotecan (CKD-602), and rubitecan.

21. The composition of claim 19, wherein the composition has a chemical structure selected from:

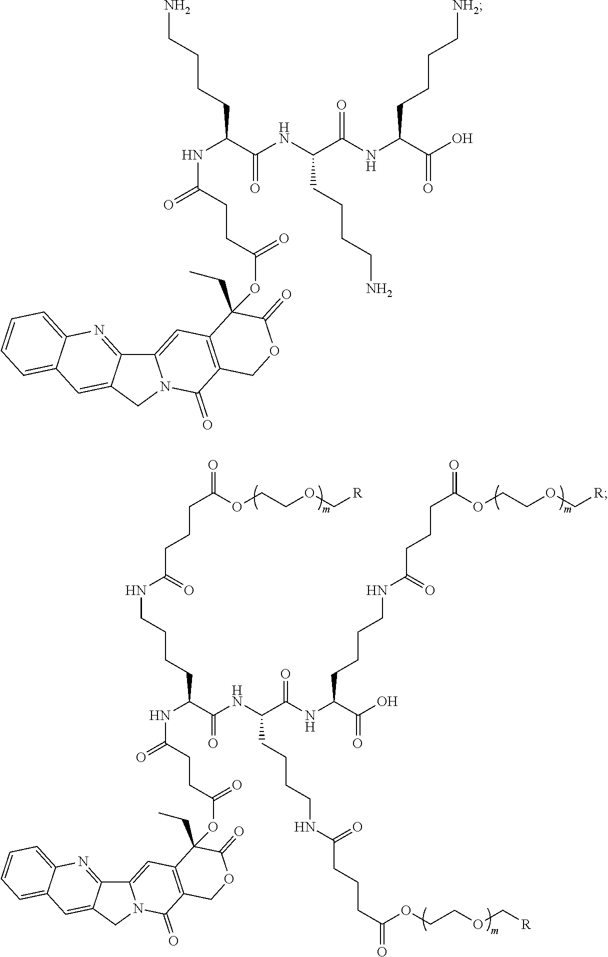

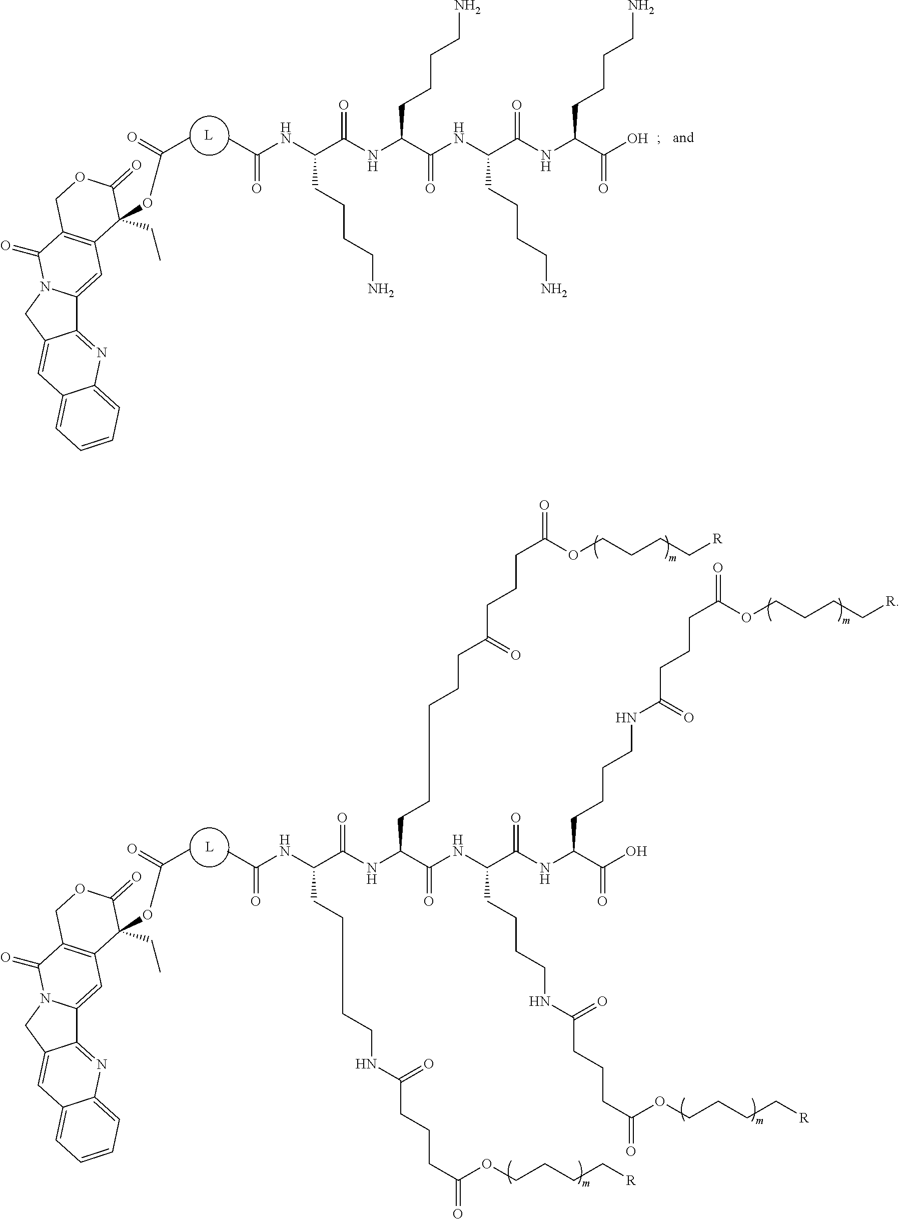

22. The composition of claim 19, wherein the composition has a chemical structure selected from:

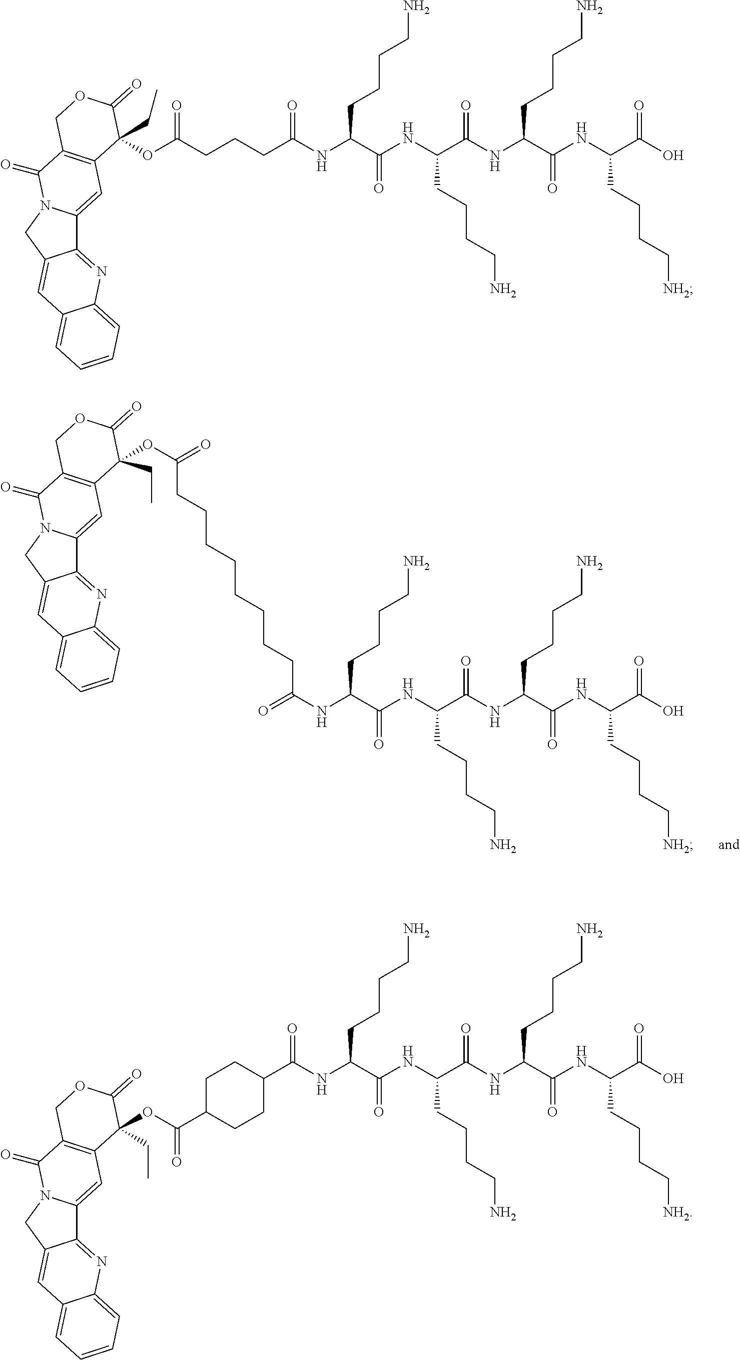 [SMILES: CC[C@]1(OC(=O)CCCC(=O)N[C@@H](CCCCN)C(=O)N[C@@H](CCCCN)C(=O)N[C@@H](CCCCN)C(=O)N[C@@H](CCCCN)C(=O)O)C(=O)OCc2c1cc1n(c2=O)Cc2cc3ccccc3nc2-1 ; CC[C@]1(OC(=O)CCCCCCCCC(=O)N[C@@H](CCCCN)C(=O)N[C@@H](CCCCN)C(=O)N[C@@H](CCCCN)C(=O)N[C@@H](CCCCN)C(=O)O)C(=O)OCc2c1cc1n(c2=O)Cc2cc3ccccc3nc2-1 ; CC[C@@]1(OC(=O)C2CCC(C(=O)N[C@@H](CCCCN)C(=O)N[C@@H](CCCCN)C(=O)N[C@@H](CCCCN)C(=O)N[C@@H](CCCCN)C(=O)O)CC2)C(=O)OCc2c1cc1n(c2=O)Cc2cc3ccccc3nc2-1]

23. The composition of claim 1, further comprising a buffer.

24. The composition of claim 23, wherein the buffer comprises a borate buffer.

25. The composition of claim 24, wherein the borate buffer comprises a nano-confined borate buffer.

26. A method for treating a disease, disorder, or condition in a subject, the method comprising administering a composition of claim 1 to a subject in need of treatment thereof.

27. The method of claim 26, wherein the disease, disorder, or condition is selected from a cancer, an infection, and an inflammation.

28. The method of claim 27, wherein the cancer is selected from breast cancer, ovarian cancer, colon cancer, stomach cancer, non-small cell lung cancer (NSCLC), a glioblastoma, and Kaposi sarcoma.

29. The method of claim 26, wherein the therapeutic agent is released by ester hydrolysis in vivo.

30. A kit comprising a composition of claim 1 and instructions for use.

31. The kit of claim 30, further comprising one or more components selected from one or more solvent or buffers, one or more vials, one or more syringes, and instructions for use.

32. A sealant comprising the composition of claim 1.
